SEQUENCE LISTING (15 sequences)

SEQ ID NO: 1 - protein, 6 aa, Artificial Sequence
  Synthetic peptide
  X (1)..(1): I or V
  X (2)..(2): E, Q or M
  X (3)..(3): Any amino acid
  X (5)..(5): A, S, W or R
  X (6)..(6): G, L or R
XXXDXX

SEQ ID NO: 2 - protein, 6 aa, Artificial Sequence
  Synthetic peptide
  X (3)..(3): P or F
  X (5)..(5): A, S, W or R
  X (6)..(6): G, L or R
IEXDXX

SEQ ID NO: 3 - protein, 6 aa, Artificial Sequence
  Synthetic peptide
  X (1)..(1): I or V
  X (2)..(2): E, Q or M
  X (3)..(3): Any amino acid
  X (5)..(5): A, S, W or R
  X (6)..(6): G, L or R
XXXDXX

SEQ ID NO: 4 - protein, 6 aa, Artificial Sequence
  Synthetic peptide
  X (3)..(3): P or F
  X (5)..(5): A, S, W or R
  X (6)..(6): G, L or R
IEXDXX

SEQ ID NO: 5 - protein, 6 aa, Artificial Sequence
  Synthetic peptide
  X (5)..(5): A, S, W or R
  X (6)..(6): G, L or R
IEPDXX

SEQ ID NO: 6 - protein, 6 aa, Artificial Sequence
  Synthetic peptide
  X (5)..(5): A, S or W
  X (6)..(6): G or L
IEPDXX

SEQ ID NO: 7 - protein, 6 aa, Artificial Sequence
  Synthetic peptide
IEPDAG

SEQ ID NO: 8 - protein, 6 aa, Artificial Sequence
  Synthetic peptide
IEPDSG

SEQ ID NO: 9 - protein, 6 aa, Artificial Sequence
  Synthetic peptide
IEPDSL

SEQ ID NO: 10 - protein, 6 aa, Artificial Sequence
  Synthetic peptide
IEPDWL

SEQ ID NO: 11 - protein, 6 aa, Artificial Sequence
  Synthetic peptide
IEPDWG

SEQ ID NO: 12 - protein, 6 aa, Artificial Sequence
  Synthetic peptide
IEPDAL

SEQ ID NO: 13 - protein, 6 aa, Artificial Sequence
  Synthetic peptide
IEFDAL

SEQ ID NO: 14 - protein, 6 aa, Artificial Sequence
  Synthetic peptide
IEPDWR

SEQ ID NO: 15 - protein, 6 aa, Artificial Sequence
  Synthetic peptide
IEPDRL

(19) United States
(12) Patent Application Publication
(10) Pub. No.: US 2024/0376517 A1
(43) Pub. Date: Nov. 14, 2024

(54) GRANZYME B DETECTION

(71) Applicant: THE UNIVERSITY COURT OF THE UNIVERSITY OF EDINBURCH, Edinburgh (GB)

(72) Inventors: Marc VENDRELL, Edinburgh (GB); Jamie I. SCOTT, Edinburgh (GB); Doireann GORDON, Edinburgh (GB)

(21) Appl. No.: 18/568,921

(22) PCT Filed: Jun. 13, 2022
(86) PCT No.: PCT/GB2022/051477

§ 371 Date: Dec. 11, 2023

(30) Foreign Application Priority Data
Jun. 11, 2021 (GB) 2108355.5

(51) Int. Cl. C12Q 1/37; C07K 7/06; G01N 33/543; G01N 33/58

(52) U.S. Cl. CPC C12Q 1/37; C07K 7/06; G01N 33/54346; G01N 33/582

(57) ABSTRACT

The present disclosure relates to the development of novel cleavable peptide probes for use in detecting granzyme B, as well as methods for using the probes in medical and biological settings. The probes may comprise a fluorescent moiety conjugated or bound to the peptide, and quencher moiety conjugated or bound to the peptide and upon cleavage of the peptide, the fluorescent moiety becomes dequenched.

### FIELD

The present disclosure relates to the development of novel probes for use in detecting granzyme B, as well as methods for using the probes in medical and biological settings.

### BACKGROUND

The response of CD8+ T cells is one of the main immune mechanisms to protect the human body against cancer. The presence of CD8+ T cells in tumors is indicative of a favourable prognosis in cancer patients; however, there is high variability in how patients respond to immunotherapies. Clinical imaging can measure the size of tumors and, to some extent, the infiltration of CD8+ T cells but they cannot provide readouts on how efficiently the immune system is responding against cancer cells. This limitation hinders the evaluation of new drugs and the personalised optimisation of immunotherapies to minimise off-target toxicity. Chemical approaches to directly measure the activity of tumor-infiltrating T cells would provide reliable indicators of response to treatment and accelerate the screening of anticancer drugs.

Standard methods for in vitro monitoring CD8+ T cell cytotoxicity employ LDH, MTS or ⁵¹Cr release assays. These assays report bulk cytotoxicity rather than T cell-specific anticancer responses. The activity of CD8+ T cells can be indirectly monitored by measuring the concentration of extracellular cytokines and membrane proteins (e.g., CD107a) using antibodies. These methods assess T cell function but do not directly report on cancer cell death, thus are not biomarkers of the immune killing capacity in tumors. The chemical design of activity-sensing reporters of granzyme B (GzmB) represents an effective strategy for monitoring the cytotoxic activity of CD8+ T cells in cancer. GzmB is a serine protease that is stored inactive in T cells until antigen-driven recognition prompts its release and activation inside cancer cells. Probes for detecting GzmB include antibodies and fusion proteins, which do not distinguish between the active and inactive forms of the enzyme,^{1,} and activatable constructs based on the Ile-Glu-Pro-Asp (IEPD) sequence first described by Thornberry et al.² Some examples of the latter include nanoprobes for urinanalysis.^{3,4} All these constructs rely on a chemical scaffold with limited catalytic efficiency (e.g., Vₘₐₓ in the range of pmol min⁻¹ and k_{cat}/K_{M} ratios around mM⁻¹ s⁻¹, see Table 1).

It is amongst the objects of the present disclosure to obviate and/or mitigate one or more of the aforementioned disadvantages.

### SUMMARY

The present disclosure is based in part on the development of probes, which are cleavable by Granzyme B and which upon cleavage generate a cleaved peptide, which is detectable, such as by an increase in signal, change in one or more physicochemical properties or increase in signal to noise ratio.

In a first aspect, there is provided a probe for use in detecting granzyme B, the probe comprising a granzyme B cleavable peptide conjugated or bound to a detectable moiety, wherein the granzyme B cleavable peptide comprises, consists essentially of, or consists of a hexapeptide sequence having the sequence:

P4-P3-P2-P1-X1-X2

wherein P4 is I or V; P3 is E, Q or M; P2 is any amino acid; P1 is D, X1 is A, S, W, or R; and X2 is G, L, or R.

The peptide is cleaved between the P1 and X1 amino acid residues. Throughout this disclosure, the conventional one-letter amino acid code is used to define amino acids.

Optionally, the hexapeptide sequence comprises, consists essentially of, or consists of the sequence:

I-E-P/F-D-X1-X2

wherein X1 is A, S, W, or R; and X2 is G, L, or R.

For the avoidance of doubt, P/F is understood to mean P or F.

The methods described herein may be used in a qualitative or quantitative sense. Thus, in certain embodiments, the methods may be used in the determination of granzyme B activity.

Following cleavage of the peptide, a cleaved peptide comprising the detectable moiety is generated, which may be detected. A variety of detectable moieties may be envisaged including, radiometric, paramagnetic contrast agents, paramagnetic or superparamagnetic particles and optically detectable moieties. In one teaching, the detectable moiety is initially quenched when conjugated or bound to the peptide. However, following cleavage of the peptide by granzyme B, dequenching of the initially quenched signal occurs and a detectable increase in signal and change in physiochemical properties may be observed.

In accordance with the present disclosure, it is possible to discern the cleaved from the non-cleaved peptides. One method envisaged employs the use of a quenching moiety which is designed to quench a signal in an intact peptide, but which is unquenched following peptide cleavage. It is also possible to use detectable signals, which are not quenched/dequenched. In one embodiment, the non-cleaved peptide can be internalised by a cell and cleavage takes place internally, so that target cells with Granzyme B display an increase in signal (e.g. fluorescence) and/or change in physiochemical properties. In another embodiment it is envisaged that a cleaved peptide may be internalised by a cell, whereas a non-cleaved peptide is not. In this manner, the cleaved peptide may be detected following cell internalisation. The non-cleaved peptide may have a moiety or overall negative charge, which prevents or reduces internalisation by a cell. However, following peptide cleavage, the cleaved peptide does not comprise the moiety or negative charge, such that it can be internalised and the cleaved peptide detected.

The probes of the present disclosure may be highly specific for granzyme B. In this regard, highly specific means that the probes are more specific for granzyme B, than other enzymes, such as other caspases, including caspase-3 and granzyme A. Typically, the probes of the present disclosure display a K_{M} of less than 30 μM, 25 μM, 20 μM, 15 μM, or 10 μM for granzyme B and/or K_{cat}/K_{M} value of greater than 1×10⁴, 1×10⁵, 1×10⁶, or 1×10⁷.

In one teaching, the signal to be detected may be an optical signal. Optical signals can be detected by a variety of methods known in the art, including visually, spectrophotometrically and the use of optical sensors, including CCD and CMOS sensors, photodiodes and the like. Any suitable optical detection method may be employed and this should not be seen as limiting.

In accordance with one teaching of the disclosure, the signal moiety may be a fluorescent moiety, which is initially quenched by the use of a quencher and the method of fluorescence resonance energy transfer (FRET). Fluorescent signal detection may be an increase in fluorescence intensity, or an alteration in fluorescence lifetime, for example. Fluorescence may be detected, for example by, fluorescence spectroscopy, fluorescence microscopy, confocal fluorescence microscopy, fluorescence image analysis, flow cytometry, laser scanning cytometry, plate multi-well fluorescence reader, or a scintillation counter.

The principle of FRET, also known as Förster resonance energy transfer (FRET), resonance energy transfer (RET), and electronic energy transfer (EET), is based on the transfer of energy from an excited donor dye to an acceptor dye or quenchers that are located in spatial proximity. Dark acceptors or quenchers are substances that absorb excitation energy from a fluorophore and dissipate the energy as heat; while fluorescent acceptors or quenchers re-emit much of this energy as light. Depending on the fluorescence quantum efficiency of the acceptor species, the energy transferred from the donor species to the acceptor species can either undergo nonradiative relaxation by means of internal conversion thereby leading to quenching of the donor energy, or can be emitted by means of fluorescence of the acceptor species.

FRET occurs between the electronic excited states of the donor species and acceptor species when they are in sufficient proximity to each other, in which the excited-state energy of the donor species is transferred to the acceptor species. The result is a decrease in the lifetime and a quenching of fluorescence of the donor species. In one application of this principle, as applicable herein, a fluorescent moiety is positioned to be in close proximity to a quencher moiety. In this configuration, the energy from the excited donor fluorescent moiety is transferred to the acceptor quencher moiety and dissipated as heat rather than fluorescence. However, when the fluorescent moiety is not in close proximity to the quencher moiety, such as following cleavage of the peptide by Granzyme B, fluorescence of the fluorescent moiety is no longer quenched by the quencher moiety and can be detected by an increase in fluorescence intensity, for example.

Thus, in accordance with one teaching of the disclosure, there is provided a highly specific probe for use in detecting granzyme B, the probe comprising a fluorescent moiety conjugated or bound to the peptide, and quencher moiety conjugated or bound to the peptide, the peptide comprising, consisting essentially of, or consisting of a granzyme B cleavable hexapeptide sequence having the sequence:

P4-P3-P2-P1-X1-X2

wherein P4 is I or V; P3 is E, Q or M; P2 is any amino acid; P1 is D, or optionally

I E P/F DX1 X2

wherein X1 is A, S, W, or R; and X2 is G, L, or R; and
wherein following cleavage of the peptide by granzyme B, dequenching of the fluorescent moiety occurs and a detectable change in fluorescent signal may be observed.

As the present disclosure is directed to the cleavage of a peptide by granzyme B, the probes and methods described herein are concerned with the detection of enzymatically active granzyme B. Thus, the present disclosure may distinguish over other teachings where granzyme B may not be active. For example, substrate binding, without enzymatic cleavage of a substrate would not indicate if the binding molecule was enzymatically active. Moreover, methods, which employ an antibody to bind granzyme B, will not necessarily distinguish between active and non-active forms of granzyme B. Thus, advantageously, the present invention is able to identify active granzyme B as opposed to simply the presence of granzyme B, which could include inactive forms. Moreover, the present methods may be used in combination with other methods for detecting total granzyme B levels. In this manner, it may be possible to calculate a ratio of total granzyme B to active granzyme B, which would also permit identification of the amount of inactive enzyme.

The detectable moiety, such as a fluorescent moiety may be conjugated to the peptide by any suitable method. Typically, the detectable moiety will be conjugated to the peptide by way of a covalent bond. For example, the detectable moiety may be conjugated to the peptide by way of an amide bond. The detectable moiety may be conjugated anywhere along the peptide sequence, but in one embodiment, the detectable moiety is covalently bonded to the N or C terminal amino acid of the peptide, such as by way of an amide bond. In one embodiment, the signal-generating moiety is covalently bonded to the N terminal amino acid. Conjugation/covalent attachment via amino acid side chains, is also envisaged. The signal-generating moiety may be directly conjugated/covalently bonded to the peptide or through a linker molecule between the detectable moiety and the peptide.

When employed, the quencher moiety may be conjugated or bound to the peptide in a similar manner to the detectable moiety as described above. However, the quencher moiety is not generally conjugated or bound to the same location on the peptide as the detectable moiety. In one embodiment, the detectable moiety is conjugated or covalently bonded to the N or C terminal amino acid, with the quencher moiety conjugated or covalently bonded via the C or N terminal amino acid respectively.

The detectable moiety, fluorescent moiety and/or quencher moiety may be indirectly conjugated to the peptide. For example, in one teaching, the fluorescent moiety and/or quencher moiety may be conjugated, bound and/or embedded within a nanoparticle(s), such as a latex nanoparticle, with the nanoparticle(s) bound to the peptide.

When employed, suitable linker molecules include aliphatic, such as alkyl, alkenyl, or polyether chains, with optionally C₂-C₂₄ repeating units. The aliphatic molecules may include one or more carboxylic and/or amino groups, for example. Suitable aliphatic linkers include aliphatic diamines e.g. 1,2-diaminoethane up to 1,10-diaminodecane and pegylated diamines e.g. 2,2′-Oxydiethanamine up to 1,8-Diamino-3,6-dioxaoctane.

Alternatively, one or more naturally or non-naturally occurring amino and/or imino acids may be used as linking groups

Fluorescent moieties suitable for use in the present invention can include a single molecule or molecular dye. Dyes useful for this invention include fluorescent, hydrophobic dyes that fluoresce in a range from 400 to 1000 nm. Classes of dyes include, but are not necessarily limited to oxonol, pyrylium, Squaric, croconic, rodizonic, polyazaindacenes or coumarins, scintillation dyes (usually oxazoles, benzothiadiazoles and oxadiazoles), aryl- and heteroaryl-substituted polyolefins (C2-C8 olefin portion), merocyanines, Rhodamines, sulphocyanines, carbocyanines, phthalocyanines, oxazines, carbostyryl, porphyrin dyes, dipyrrometheneboron difluoride dyes, aza-dipyrrometheneboron difluoride dyes, and oxazine dyes. Commercially available fluorescent dyes may be obtained from Thermofisher, Sigma Aldrich, for example.

Exemplary quencher dye compounds suitable for use in the present disclosure can include a single molecule or molecular dye. Suitable quencher dyes include e.g. DABCYL and QSY-series from Molecular Probes (www.probes.com), Dark Cy-dyes from Amersham Biosciences (www.amershambiosciences.com), Eclipse Dark Quencher dyes from Epoch Biosciences (www.epochbio.com), Black Hole Quencher dyes from Biosearch Technologies (www.biosearchtech.com), DYQ-dyes from Dyomics (www.dyomics.com), Black Berry Quenchers from Berry & Associates (www.berryassoc.com), QXL quenchers from AnaSpec, Inc. and ElleQuencher from Oswel (www.oswel.com). Additional quenchers include Methyl Red, Iowa Black FQ, Iowa Black RQ (Integrated DNA Technologies), IRDye QC-1 (Licor) and Si-Rhodamine-based NIR dark quenchers (Mycohin et al., J. Am. Chem. Soc. 2015, 137, 14, 4759-4765).

The fluorescent moieties and quencher dyes need to be sufficiently close when bound to the peptide, in order to ensure suitable quenching of any fluorescent signal, before peptide cleavage. As shown herein, when hexapeptides are employed and the fluorescent and quencher moieties are bound to each end of the peptide, sufficient quenching is observed. The skilled addressee can easily test the quenching ability for longer peptides and whether or not the fluorescent and/or quencher moieties can be bound to the peptide ends, or if the fluorescent and/or quencher moieties may have to be conjugated or bound via an internal group of the peptide

In one embodiment, the peptide sequence comprises or consists of the sequence:

I E P D X1 X2, with the definitions of X1 and X2 as defined above.

In one embodiment, X1 is A, S, or W and/or X2 is G, or L.

In one embodiment, the peptide is selected from the group consisting of

I E P D A G; I E P D S G; I E P D S L; I E P D W L; I E P D W G; I E P D A L; I E F D A L.

The probes of the present disclosure desirably are capable of detecting granzyme B in an amount of less than 10 nM, such as less than 1 nM, 500 pM, 250 pm, 100 pm, or less than 50 pm.

The probes of the present disclosure desirably display rapid reactivity. In this context, rapid reactivity may mean the ability to cleave a peptide using granzyme B over a short period of time. In one embodiment, this may be the percentage ability to cleave the peptide over a number of hours, such as within 2 hours. In one test described herein, a test employed was to ascertain the percentage cleavage of the peptide over a period of two hours. In some embodiments, in accordance with this test, peptides, which display 70% or higher cleavage, such as at least 75% or 80% cleavage, are desired.

The probes as described herein may be provided in a solution, or may be bound to a substrate, such as in the well of a micro titre plate, surface of a microfluidic channel, surface within a lateral flow system, or any other suitable surface employed in well-known analyte detection assays. The probes may also be contacted with Granzyme B, which is free within the bodily fluid or excretion, cell sample, or biopsy, or to Granzyme B, which has first been captured by use of a Granzyme B specific biding agent, as described herein.

Granzyme B-mediated cytotoxicity is the major mechanism by which cytotoxic T lymphocytes (CTL) and natural killer (NK) cells eliminate pathogen-infected cells (including viral pathogens such as COVID-19), transformed cancer cells, epithelial cells, such as epithelial cell associated in IBD as well as non-self cells in transplant rejection events, such as kidney, liver or lung transplants.

Thus, the probes of the present disclosure may find application in detecting immune-mediated cell death, such as cytotoxic activity of T and/or NK cells, on aberrant or undesirable cells. The aberrant or undesirable cells may be abnormally proliferating cells, e.g. malignant (i.e., cancer) or non-malignant tumour cells, Granzyme B may also be found in gut epithelial cells, virally or bacterially infected cells, as well as CD4+ T cells, mast cells, activated macrophages, neutrophils, basophils, dendritic cells, T regulatory cells, smooth muscle cells, chondrocytes, keratinocytes, type II pneumocytes, Sertoli cells, primary spermatocytes, granulosa cells, syncytial trophoblasts.

The activity of granzyme B may also be detected extracellularly. GzmB is present in the serum of healthy individuals (approx. 20-40 pg/mL) and is upregulated in serum of patients with HIV, Epstein Barr virus, arthritis and inflammatory bowel disease (IBD), for example. GzmB is also found in synovial fluid of rheumatoid arthritis patients, cerebrospinal fluid of multiple sclerosis patients and Rasmussen encephalitis patients. It is also found in bronchoalveolar lavage from COPD patients and those suffering from lung inflammation or pulmonary sarcoidosis, for example. GzmB is also associated with acute transplant rejection.

Thus, in one teaching, there is provided use of a probe, as described herein, in detecting a level, such as an aberrant level, of granzyme B in cells within a sample and/or bodily fluids, as discussed above. A suitable sample may include a tissue or biopsy sample. Bodily fluids, may include any suitable bodily fluid cerebral spinal fluid (CSF), whole blood, serum, plasma, cytosolic fluid, urine, faeces, stomach fluids, digestive fluids, saliva, nasal or other airway fluid, vaginal fluids, or semen, Most typically, the biological fluid is blood, plasma or serum. Typically, the sample is obtained from any suitable animal, most typically a mammal, such as a human. In one embodiment, the disease/condition to be detected is IBD and the sample is a stool, or blood (e.g. serum or plasma) sample. In one embodiment, the condition is acute transplant rejection and the sample is a urine (for kidney), blood (serum or plasma), or lavage (for lung) sample. It is to be understood that an aberrant level may be discerned in comparison to a normal or baseline level of granzyme B, as detected, for example, from non-diseased cells or a bodily fluid from a subject not suffering from said identified diseases or conditions, in order to determine whether or not the level of granzyme B is substantially increased, or decreased (or the same), with respect to a normal or baseline level. An increased or decreased level may be understood to be at least 10%, 20%, 30%, 50%, 100%, 200% or more, of a difference with respect to a normal or baseline level.

Thus, in one embodiment, a stool sample is processed, in order to provide a supernatant comprising a portion of stool sample. The supernatant is contacted with a suitable Granzyme B binding agent, such as an anti-Granzyme B capture antibody, which may be attached or adhered to a surface, for example, in order to bind any Granzyme B, which is present in the portion of stool sample. Thereafter a peptide, as defined herein, is contacted with any Granzyme B, which is bound to the anti-Granzyme B antibody and in order to detect and optionally quantify any Granzyme B which is present in the portion of stool sample.

Other binding agents include antibody fragments, which are capable of specifically binding Granzyme B, nanobodies, aptamers, and receptors or other proteins which can specifically bind Granzyme B.

The surface may be any suitable surface, such as the surface of a slide, microplate, wall in a fluidic device and the like, but may also be the surface of a bead or micro or nanoparticle, for example, to which the binding agent is adhered, attached, or otherwise immobilised.

Cells, may be individual cells or multiple cells, as would be obtained, for example, from a tissue biopsy or sample.

In a further teaching, there is provided a method of detecting Granzyme B in a cell or bodily fluid, the method comprising contacting a probe as described hereinabove with a cell or bodily fluid sample, in situ, or in vitro, that is isolated from a subject, and detecting a level of granzyme B, by cleavage of the peptide and release of the detectable signal moiety.

The probes of the present invention may be further used in methods to detect any effect (such as a therapeutic or cytotoxic effect) of a pharmaceutical agent on a population of cells, in vitro, ex vivo, or in vivo.

### DETAILED DESCRIPTION

The present disclosure will now be further described by way of example and with reference to the Figures, which show:

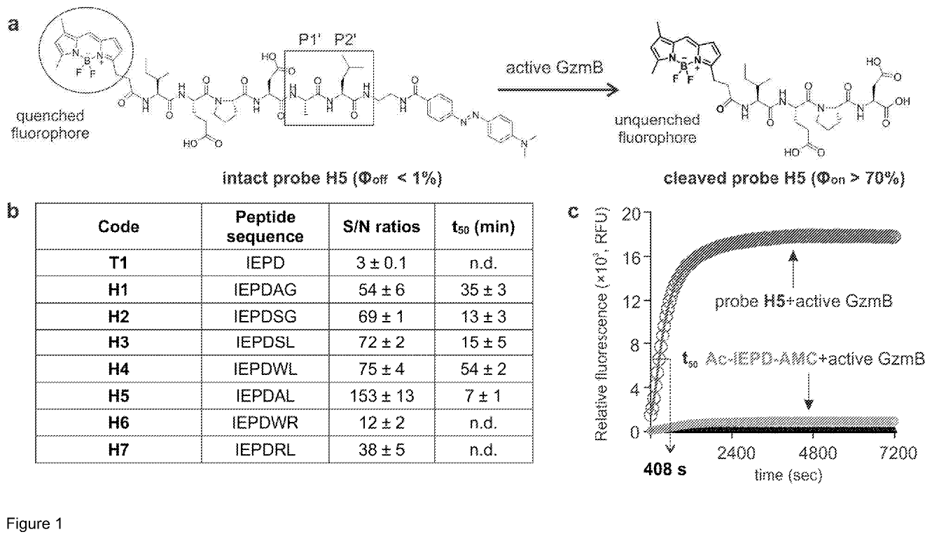

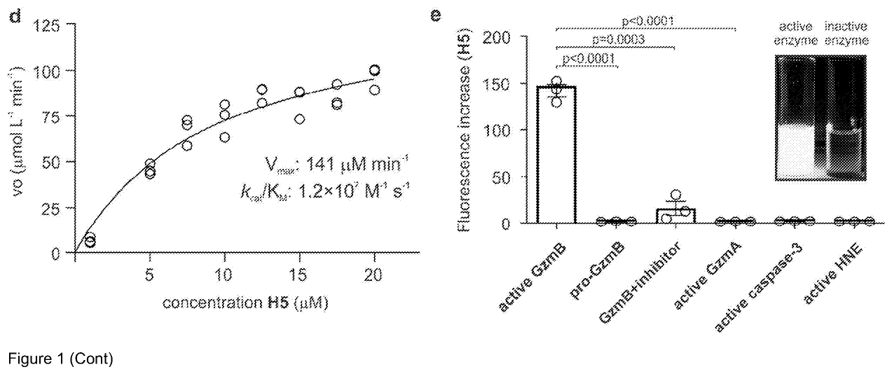

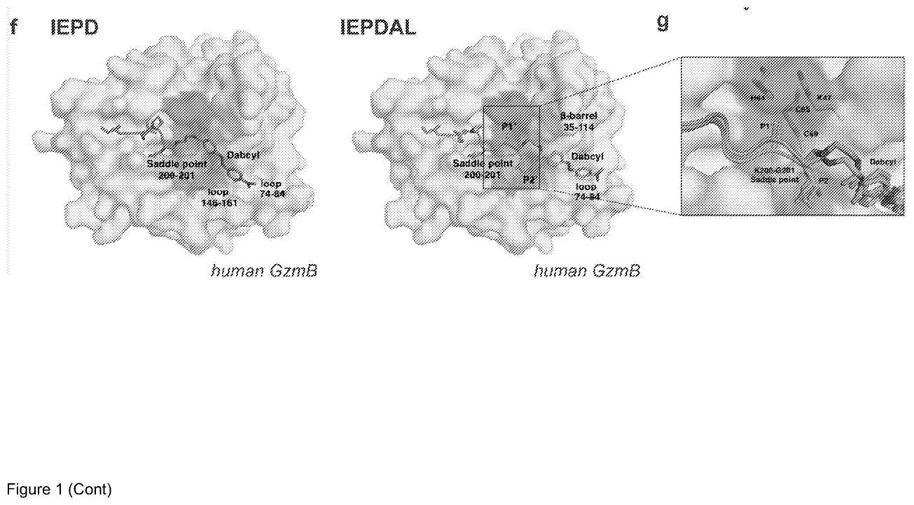

FIG. 1. Shows the hexapeptide H5 achieves high reactivity and selectivity for GzmB by accessing a unique binding pocket. a) Fluorogenic hexapeptide H5 and fluorescence quantum yields of intact and cleaved probe. b) Tetra- and hexapeptide sequences, fluorescence increases and t₅₀ values upon incubation with hGzmB. Data as means±SEM (n=3). c) Time-course fluorescence of H5 (25 μM, dark grey, 510 nm) and Ac-IEPD-AMC (25 μM, light grey, 450 nm) after incubation with hGzmB (20 nM) at 37° C. Peptide H5 alone (25 μM, black, 510 nm). Data as means±SEM (n=5). d) Fluorescence changes of peptide H5 (25 μM) after incubation with proteases (20 nM) at 37° C. for 60 min. Data as means±SEM (n=3). e) Cleavage rate of peptide H5 by hGzmB (20 nM) as a function of substrate concentration. (n=3). f) Representative binding mode of IEPD-Dabcyl in T1 (left) and IEPDAL-Dabcyl in H5 (right) from the MD simulations. g) Detailed interactions at P1′ and P2′ sites for the peptide H5 with overlaid structures from 3 independent runs.

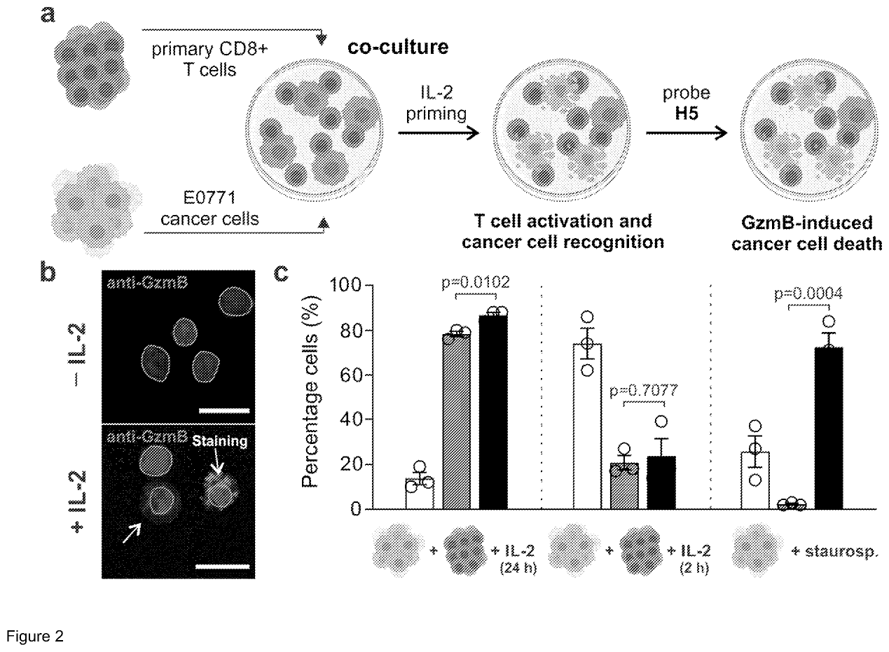

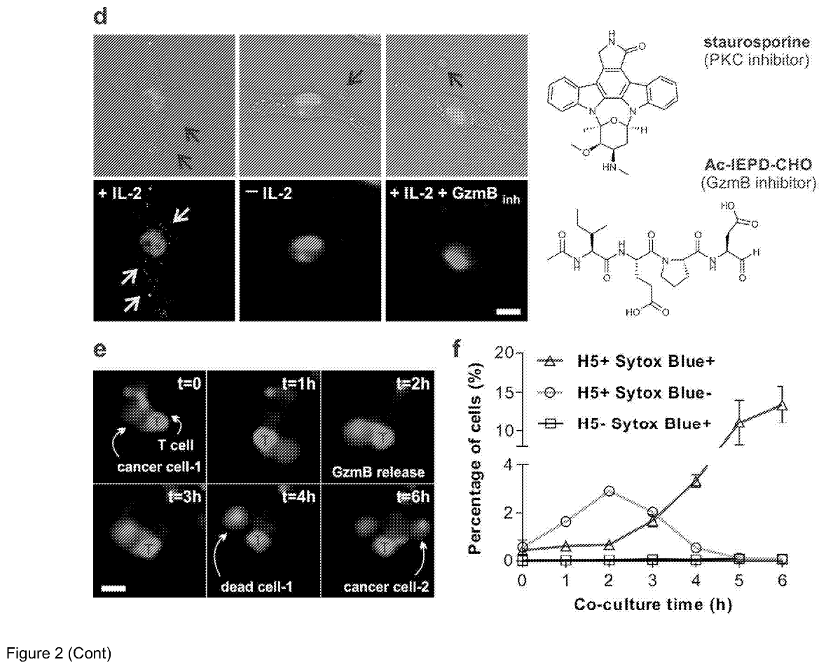

FIG. 2. Probe H5 detects GzmB-mediated anticancer activity of CD8+ T cells. a) Schematic procedure for co-culture assays. b) Representative microscopy images of CD8+ T cells before/after reinvigoration and staining by anti-GzmB (white arrows show fluorescence signal) and Hoechst 33342 (outline of nucleus drawn in grey) (n=3). Scale bar 10 μm. c) Flow cytometry 0 of E0771 cells in co-culture with active (IL-2, 24 h) or inactive (IL-2, 2 h) CD8+ T cells, or alone with staurosporine (1 μM, 1 h): viable (white), H5-stained (grey), apoptotic (black). (n=3). d) Confocal microscopy images of mKate-expressing E0771 cancer cells (nucleus displays large circular signal) stained with H5 ( ) in co-culture with active T cells (left), non-active T cells (centre) or active T cells plus Ac-IEPD-CHO (right). Black arrows highlight T cells, white arrows highlight H5-stained intracellular GzmB puncta. Scale bar 10 μm. e) Time-course fluorescence microscopy of OT-I CD8+ T cells (highlighted with letter T) killing OVA-EL4 cancer cells in the presence of H5 (visible after t=2 h). Scale bar 7 μm. f) Flow cytometric analysis of OVA-EL4 cancer cells from experiments in e (n=3). P values from two-tailed t tests.

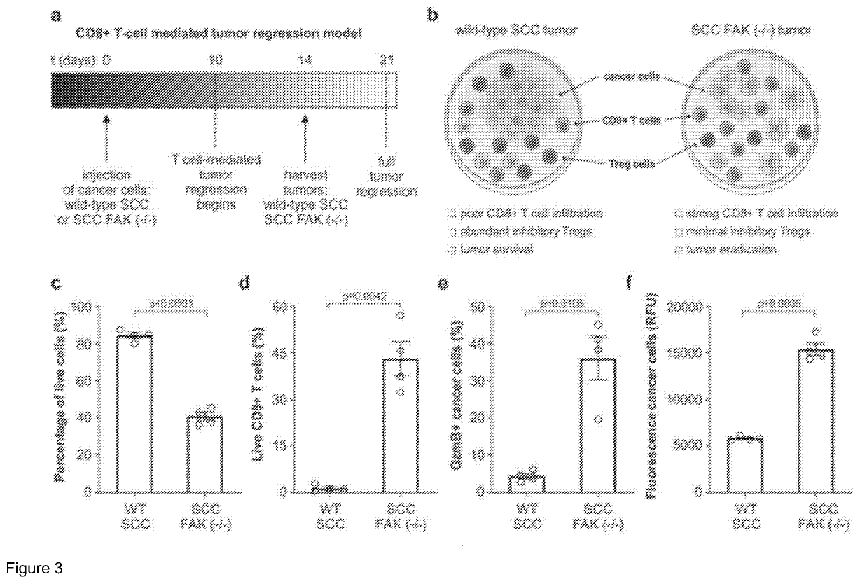

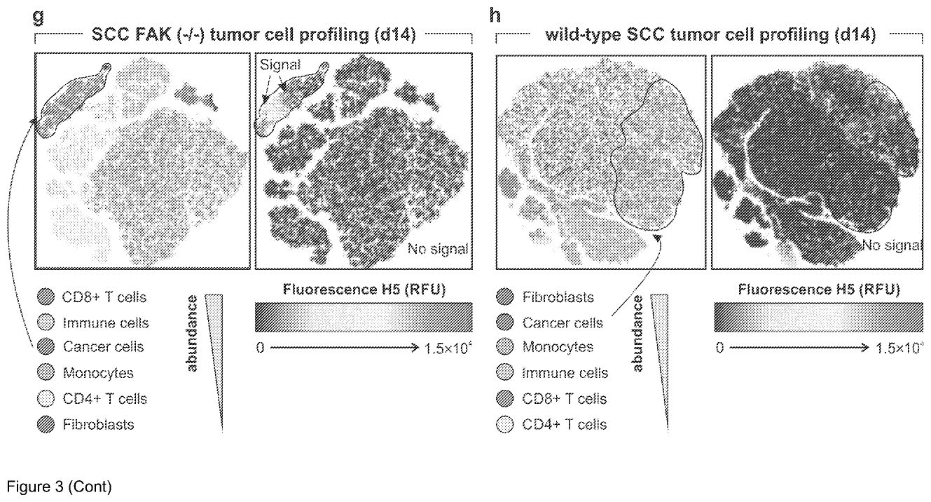

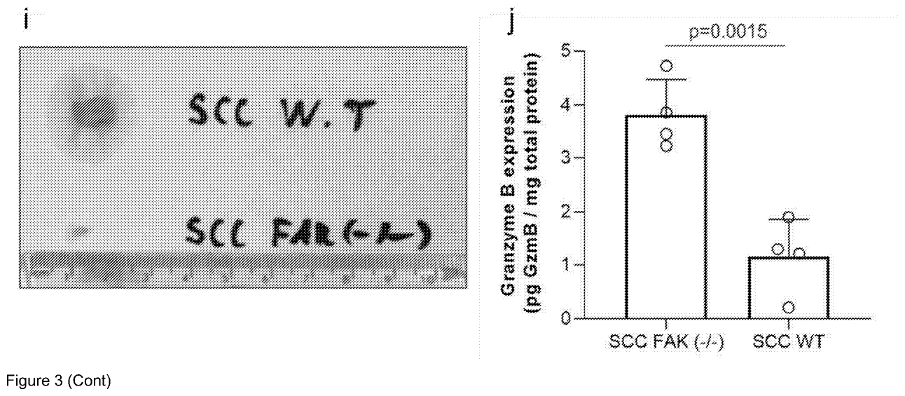

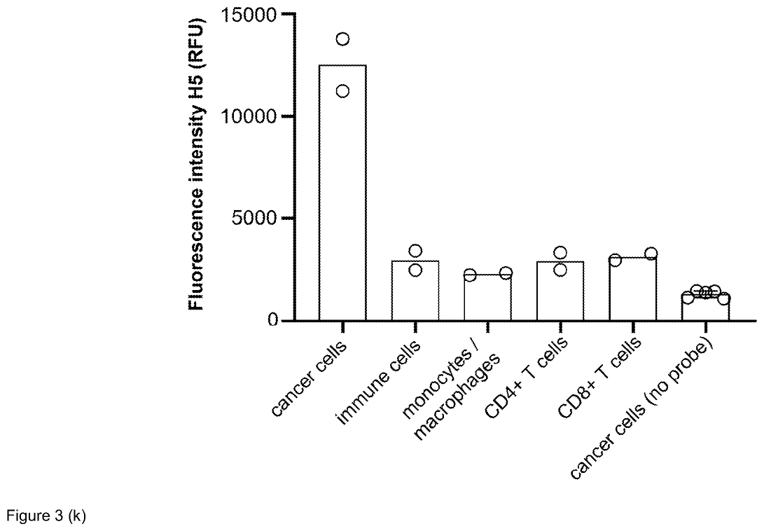

FIG. 3. Probe H5 detects T cell-mediated tumor regression in a mouse model of squamous cell carcinoma. a) Experimental timeline of the CD8+ T cell-mediated tumor regression model. b) Cell populations found in wild-type SCC and SCC FAK (−/−) tumors. c-f) SCC and SCC FAK (−/−) cells were injected into FVB immunocompetent mice (1×10⁶ cells/mouse) and tumors were harvested on day 14. Flow cytometry of wild-type SCC and SCC FAK (−/−) tumors for cell viability with live/dead stain (c); CD8+ T cell infiltrates with anti-CD8-PE (d); percentage of GzmB-positive SCC cancer cells by staining with H5 (e); fluorescence intensity of H5 inside SCC cancer cells (525 nm) (f). (n=4). SCC FAK (−/−) tumors (g) and wild-type SCC tumors (h) (ex vivo stained with 5 μM compound H5) were analyzed by flow cytometry and presented as pseudo-colored two-dimensional tSNE (t-distributed stochastic neighbor embedding) plots to determine the distribution of the probe in different cell populations (left) and the fluorescence intensity of H5 staining (right) (n=4) i) Representative picture of harvested SCC7.1 and SCC7.1 FAK−/− tumors on day 14 post-injection of cancer cells. j) Quantification of GzmB levels in both tumours by ELISA after tissue disaggregation and protein extraction. GzmB levels were normalized to total protein amount as measured by the BCA method. Data presented as mean values±SD (n=4). P values were obtained from two-tailed t tests. j) Fluorescence emission in multiple cell subsets. Data presented as mean values±SEM (2 independent experiments).

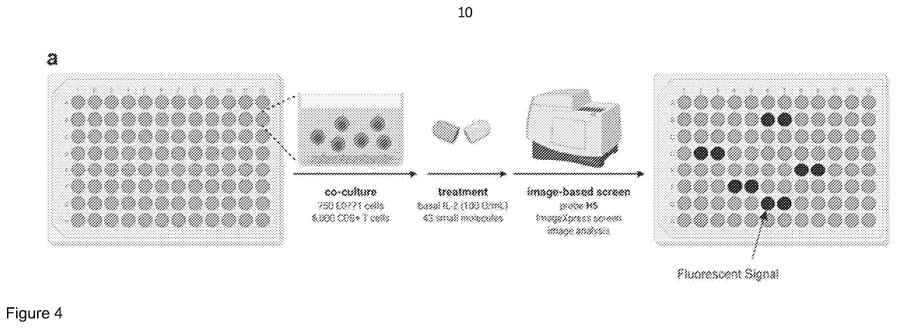

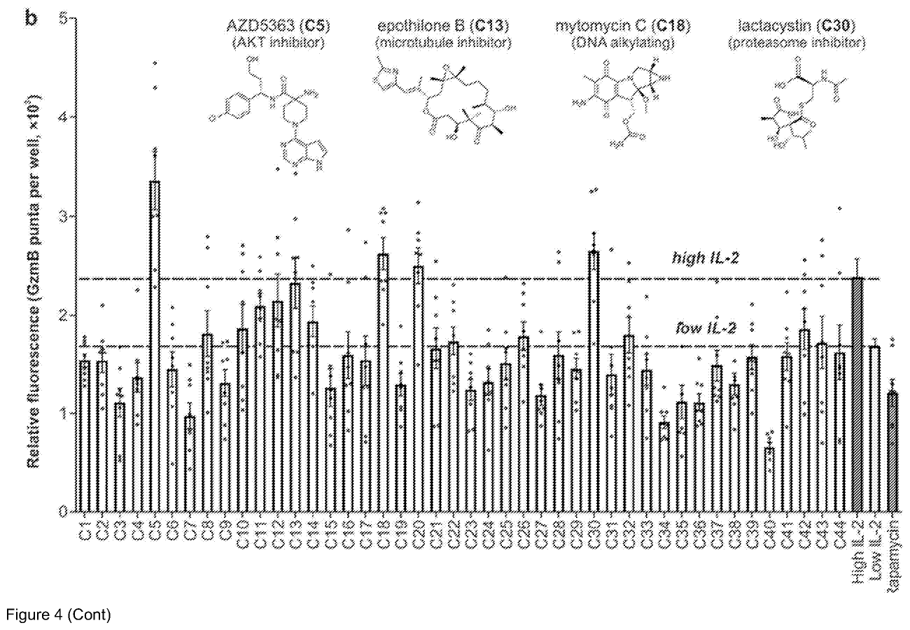

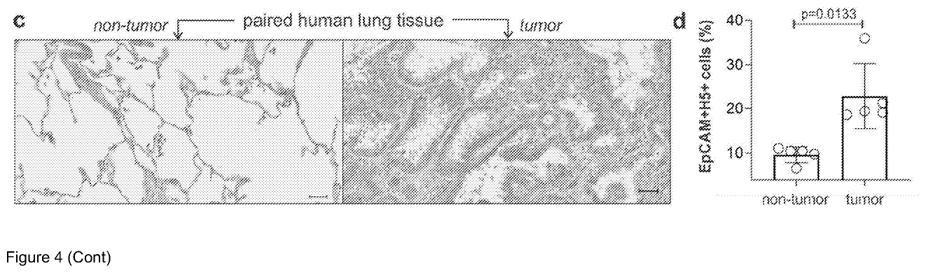

FIG. 4. Probe H5 detects immunomodulatory action in phenotypic screens and T cell cytotoxic activity in human lung cancer biopsies. a) Experimental protocol of the phenotypic screen. b) Fluorescence intensity of probe H5 in co-cultures of E0771 cells and IL-2-activated CD8+ T cells after incubation with small molecules (C1-C44. High IL-2 used as a positive control for invigorated CD8+ T cells, and rapamycin used as a negative control. Probe H5 was incubated for 1 h and fluorescence images were acquired. (n≥8). Chemical structures of drugs showing H5 fluorescence signals above the positive control. c) Representative H&E microscope images of healthy (left) and cancerous (right) regions in biopsies taken from lung adenocarcinoma patients. d) Cytometry analysis of EpCAM+H5+ cells in paired (healthy vs cancerous) biopsies from cancer patients after incubation with probe H5. (n=3).

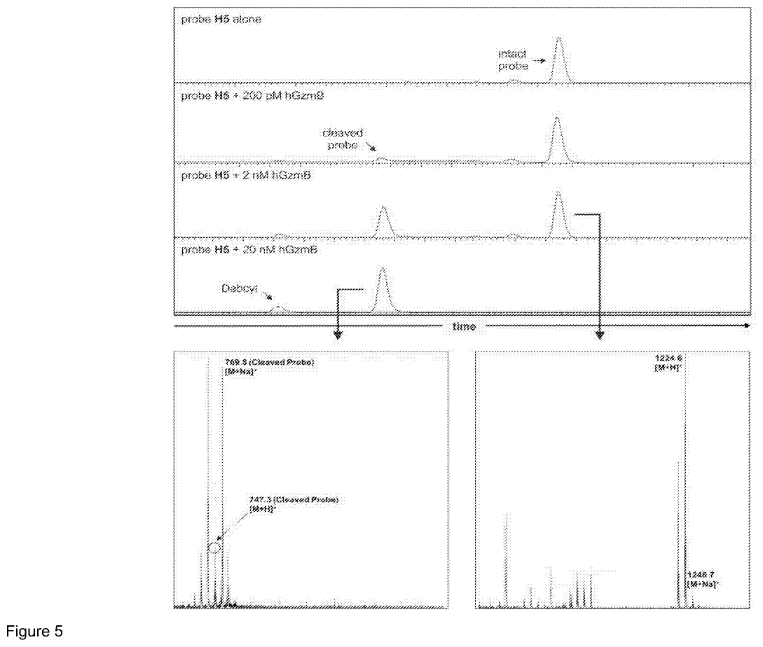

FIG. 5. HPLC traces of the probe H5 before and after reaction with hGzmB. HPLC chromatogram and mass spectrometry analysis of the probe H5 before and after reaction with increasing concentrations of hGzmB. Mcalc. (intact probe): 1246.6 [M+Na⁺]; Mcalc. (cleaved probe): 747.2 [M+H⁺].

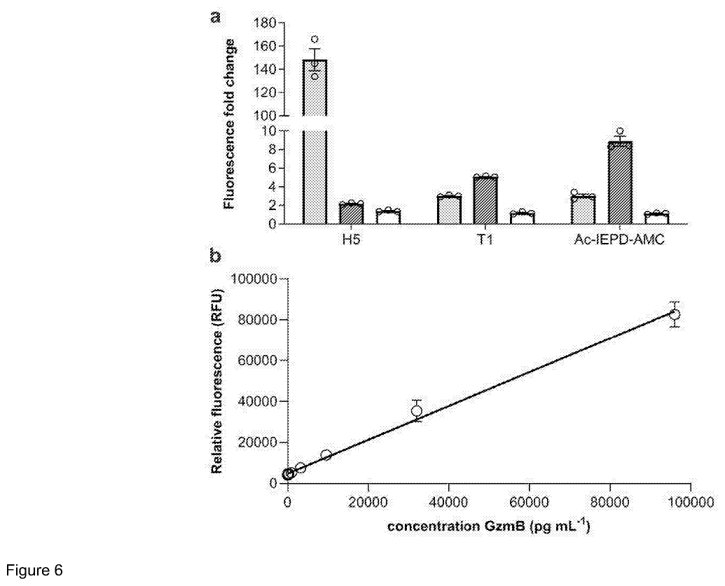

FIG. 6. Comparative enzyme selectivity and limit of detection for the hexapeptide H5. a) Fluorescence response for the fluorogenic peptides H5, T1 and Ac-IEPD-AMC after incubation with hGzmB (light gray bars), human caspase-3 (dark gray bars) and hGzmA (white bars) (n=3). b) Limit of detection of hGzmB by fluorescence emission (530 nm) of the hexapeptide H5 after reaction with increasing amounts of hGzmB (0, 9.6, 32, 96, 320, 960, 3,200, 9,600, 32,000 and 96,000 pg mL⁻¹) at 37° C. Data presented as mean values±SD (2 independent experiments with at least 3 replicates for each).

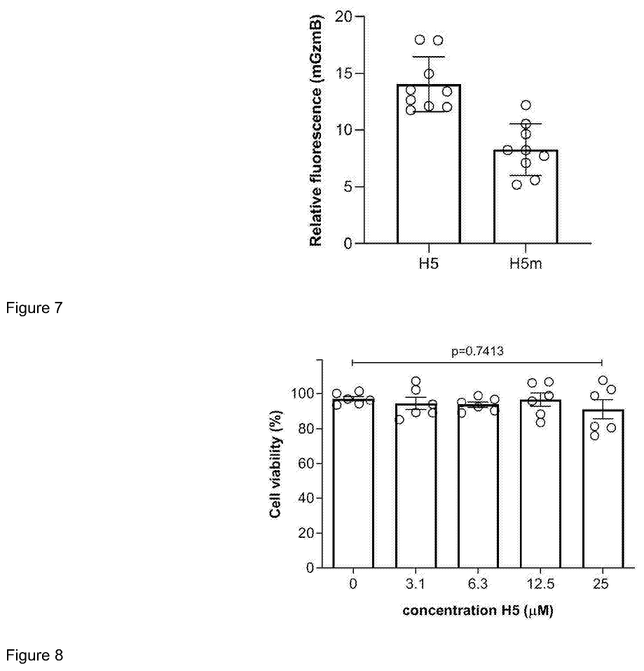

FIG. 7. Reactivity of the hexapeptides H5 and H5m against recombinant mouse GzmB. Fluorescence fold increase for the fluorogenic peptides H5 and H5m (both at 25 μM) after incubation for 90 min at 37° C. with recombinant mouse pro-GzmB (100 nM) after pre-activation with mouse cathepsin C for 4 h. Excitation/emission wavelengths: 450 nm/510 nm. Data presented as mean values±SEM (3 independent experiments including 3 separate replicates each).

FIG. 8. Cell viability assays in mKate-E0771 cancer cells. mKate-E0771 cells were plated in 96-well plates (50,000 cell well⁻¹) and incubated at the indicated concentrations of probe H5 for 1 h at 37° C. Cell viability was determined using a commercially available MTT kit (Invitrogen) with values normalized to untreated cells. Data presented as mean values±SEM (2 independent experiments with 3 replicates for each).

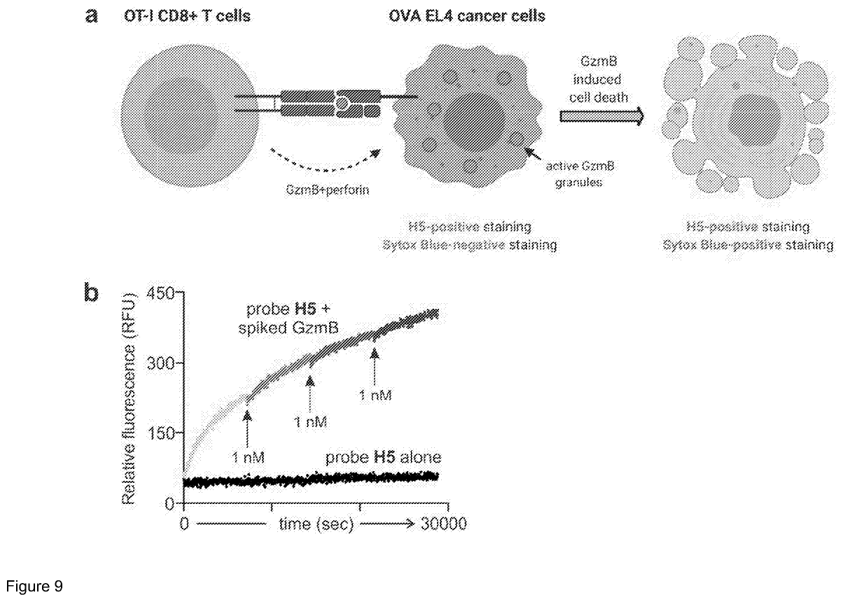

FIG. 9. Co-cultures of OT-I CD8+ T cells and OVA-EL4 cancer cells. a) Schematic cartoon of the immunological synapse between CD8+ T cells and cancer cells, highlighting the fluorescence staining from the peptide H5 (green) prior to cellular apoptosis and the dead cell marker Sytox Blue (blue) after granzyme B has initiated apoptosis. b) Longitudinal fluorescence emission of probe H5 (1 nM) upon spiking hGzmB at time 0 and at different timepoints highlighted by the arrows (1 nM increments of hGzmB) (n=3).

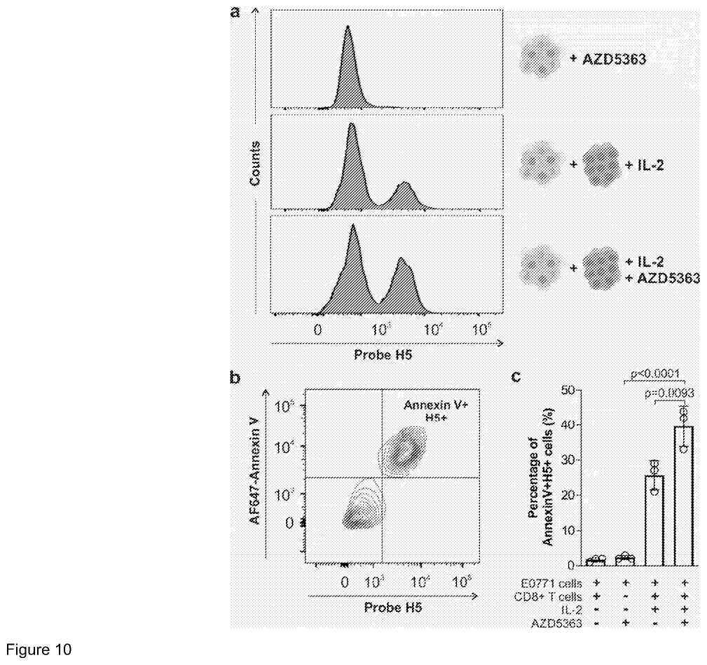

FIG. 10. Probe H5 detects CD8+ T cell mediated killing of cancer cells induced by IL-2 and AZD5363. a) Representative histograms of probe H5 staining in E0771 cells alone (50,000 cells well⁻¹) or in co-culture with CD8+ T cells (200,000 cells well⁻¹) and treatment of AZD5363 (1 μM). Excitation/emission wavelengths: 488 nm/525 nm. b) Representative flow cytometry contour plots 0 of E0771 cells (50,000 cells well⁻¹) after co-culture with murine CD8+ T cells (200,000 cells well⁻¹) and dual treatment with IL-2 (200 U mL⁻¹) and AZD5363 (1 μM). Co-cultures were stained with probe H5 (5 μM) and AF647-Annexin V (10 nM). Excitation/emission wavelengths: 488 nm/525 nm (for probe H5), 633/670 nm (for AF647-Annexin V). c) Percentages of E0771 cells that were double-stained with probe H5 and AF647-Annexin V under the experimental conditions described in a). Data presented as mean values±SEM (n=3).

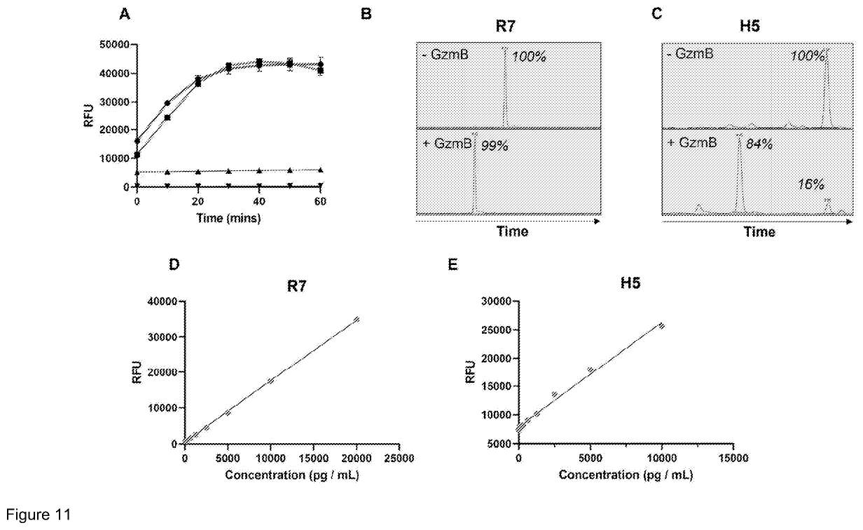

FIG. 11. A) Fluorescence response of probes H5 or R7 (25 μM, black circle and black squares, respectively) after incubation with recombinant human granzyme B (15 nM) for 1.5 h at 37° C. or without granzyme B (H5: triangle, R7: inverted triangle) in urine (n=3). HPLC chromatograms showing R7 (Panel B) and H5 (Panel C) after incubation with (below) or without (above) granzyme B in human urine for 3 hours at 37° C. D) Limit of detection analysis for human recombinant granzyme B spiked in human urine after incubation with probe H5 for 3 h at 37° C. E) Limit of detection analysis for human recombinant granzyme B spiked in human urine after incubation with probe R7 for 3 h at 37° C.

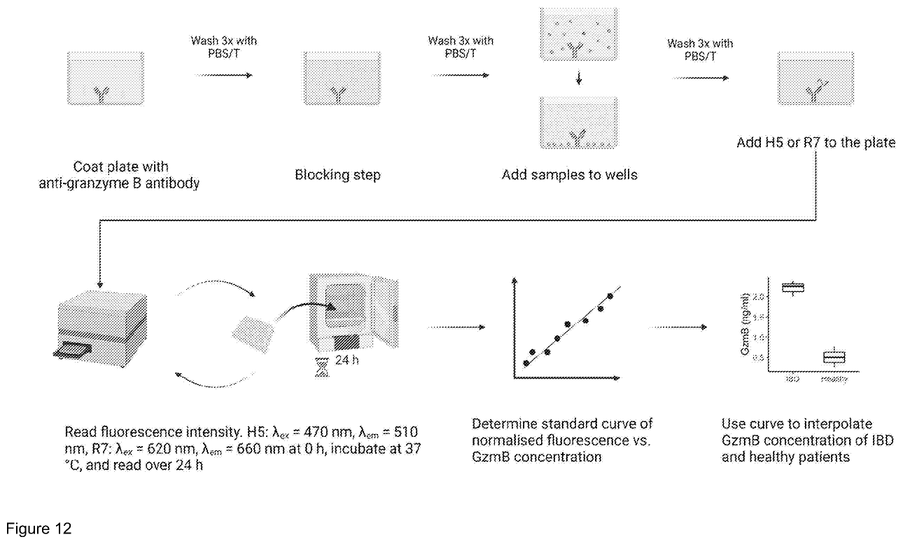

FIG. 12. Schematic representation of the in-house developed granzyme B antibody capture assay. Excitation/emission H5: 450/510 nm, R7: 620/660 nm.

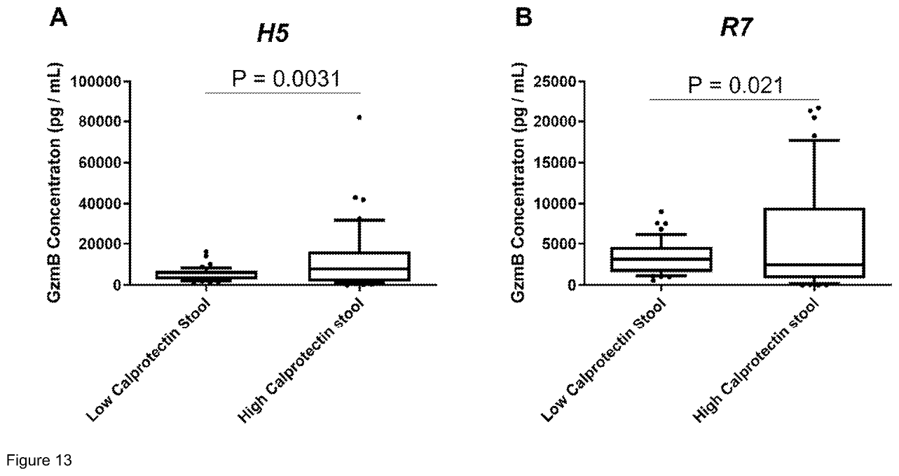

FIG. 13. Average concentration of granzyme B per group as determined by interpolation of a granzyme B calibration curve after incubation of probe H5 (A) or R7 (B) (both at 25 μM) with healthy (n=46) and IBD stool (n=48) samples for 18 h at 37° C. using the method outlined in FIG. 12. H5 λ_{exc/emi}: 450/510 nm. R7 λ_{exc/emi}: 620/660 nm Significance calculated by un-paired t test, p values indicated on graphs. High calprotectin classified as having a calprotectin level greater than 500 ug/mg of sample. Low calprotectin classified as having a calprotectin level as less than 100 ug/mg.

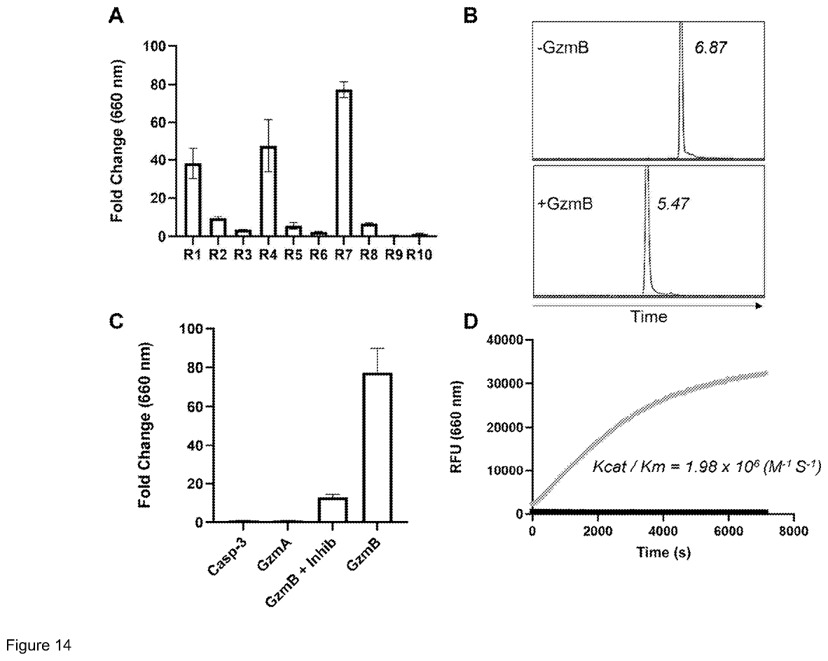

FIG. 14. A) Fluorescence fold changes of probes R1-10 (25 μM) after incubation with human recombinant granzyme B (20 nM) for 2 h at 37° C. (n=6), error bars as SEM. B) Representative HPLC spectra of probe R7 before (top) and after (bottom) incubation with granzyme B for 2 h at 37° C.). C) Fluorescence fold change of probe R7 after incubation with selected enzymes (Casp-3: Caspase-3, GzmA: Granzyme A, GzmB=Granzyme B) including granzyme B pre-inhibited with commercial inhibitor (Ac-IEPD-CHO, Enzo life sciences) (n=3) error bars as SEM. D) Fluorescence kinetic of R7 (25 μM) after incubation with (Grey) or without granzyme B (black) and associated K_{cat}/Kₘ value. (n=3). Probe Excitation: 620 nm, probe emission recorded at 660 nm.

### METHODS

### Chemical Synthesis.

### Procedure for C-Terminal Functionalization

To a solution of peptide fluorophore conjugate (1 eq) in CH₂Cl₂:DMF (1:1, 1 mL) was added benzyl(2-aminoethyl)carbamate (2 eq), Oxyma (2.5 eq) and PyOxim (2.5 eq). The reaction mixture was stirred at −20° C. for 5 min. Then DIPEA (5 eq) was added and stirring was maintained for 3 h at −20° C. The reaction was warmed to r.t. and the solvents were removed under reduced pressure. The residue was purified by semi-preparative HPLC to obtain the purified peptides.

### General Procedure for Hydrogenations

Fluorescent peptides (1 eq) and Pd/C (10%) (0.5 eq) or Pd(OH)₂/C (20%) (0.5 eq) were dissolved in 2% formic acid in MeOH (5 mL), previously purged with N₂. The reaction vessel was flushed with N₂, evacuated and filled with H₂ gas. The reaction mixture was stirred under H₂ gas at r.t. and atmospheric pressure for 2 h. Afterwards, the reaction mixture was filtered through Celite to remove the catalyst and the filtrate was evaporated under reduced pressure to isolate the deprotected peptides.

### General Procedure for Dabcyl Couplings

To a solution of Dabcyl-OSu (1.2 eq) in CH₂Cl₂:DMF (1:1, 1 mL) was added peptide (1 eq) and DIPEA (2 eq). Stirring was maintained at r.t. for 24 h. The solvents were removed under reduced pressure and the residue was dissolved in MeOH and purified by semi-preparative HPLC to obtain the final peptides.

### Benzyl(2-aminoethyl)carbamate

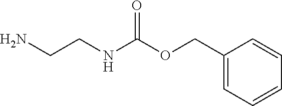 [SMILES: NCCNC(=O)OCc1ccccc1]

Benzyl chloroformate (428 μL, 3 mmol) in CH₂Cl₂ (10 mL) was added dropwise over 1 h to a solution of 1,2-diaminoethane (2 mL, 30 mmol) in CH₂Cl₂ (40 mL) at 0° C. The reaction was stirred for 1.5 h at 0° C. and then at r.t. overnight. TLC analysis (CHCl₃:MeOH, 7:3) indicated the reaction was complete. The precipitate formed in the reaction was removed by filtration and the filtrate was then washed with brine, dried over MgSO₄ and solvents were removed under reduced pressure to yield the compound as an amorphous yellow solid (570 mg, 98% yield). The crude was used in the next step without any further purification.

¹H NMR (500 MHz, CD₃OD) δ 7.44-7.25 (m, 5H), 5.09 (s, 2H), 3.22 (t, J=6.2 Hz, 2H), 2.75 (t, J=6.2 Hz, 2H).

¹³C NMR (126 MHz, CD₃OD) δ 157.7, 136.9, 128.0, 127.5, 127.4, 66.0, 42.6, 40.8.

MS (ESI+, H₂O/MeCN): [M+H⁺] calcd. for C₁₀H₁₅N₂O₂: 195.1; found: 195.3.

All spectral properties are in accordance with the literature.

### 2,5-dioxopyrrolidin-1-yl 4-((4-(dimethylamino)phenyl)diazenyl)benzoate (Dabcyl-OSu)

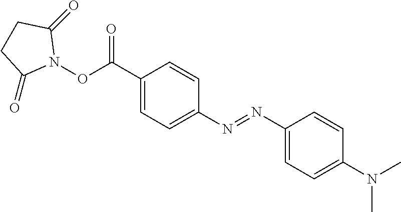 [SMILES: CN(C)c1ccc(/N=N/c2ccc(C(=O)ON3C(=O)CCC3=O)cc2)cc1]

N-hydroxysuccinimide (85 mg, 0.74 mmol), 4-((4-(dimethylamino)phenyl)diazenyl)-benzoic acid (100 mg, 0.37 mmol) and EDC HCl (142 mg, 0.74 mmol) were dissolved in a mixture of CH₂Cl₂:DMF (1:1, 10 mL) and stirred for 16 h at r.t. The solvents were then removed under reduced pressure to obtain the crude product. The crude was purified by flash column chromatography (CH₂Cl₂:MeOH, 99:1) to give the compound as an orange solid (100 mg, 88% yield).

¹H NMR (500 MHz, DMSO-da) δ 8.25-8.20 (d, J=8.6 Hz, 2H), 7.98-7.93 (d, J=8.7 Hz, 2H), 7.89-7.84 (d, J=8.7 Hz, 2H), 6.90-6.85 (d, J=8.8 Hz 2H), 3.11 (s, 6H), 2.91 (s, 4H).

¹³C NMR (126 MHz, DMSO-d) δ 170.8, 161.9, 157.0, 153.9, 143.2, 131.9, 126.2, 124.4, 122.9, 112.1, 26.0.

MS (ESI+, H₂O/MeCN): [M+H⁺] calcd for C₁₉H₁₉N₄O₄: 366.1; found: 366.0.

### Probe H5

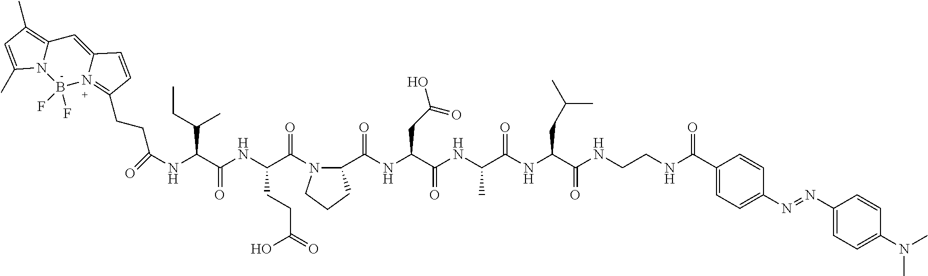

¹H NMR (500 MHz, MeOD) δ 7.98 (d, J=8.5 Hz, 2H), 7.86 (dd, J=10.2, 8.8 Hz, 4H), 7.42 (s, 1H), 7.01 (d, J=4.1 Hz, 1H), 6.86 (d, J=9.3 Hz, 2H), 6.33 (d, J=4.0 Hz, 1H), 6.22 (s, 1H), 4.68 (dd, J=8.7, 5.4 Hz, 1H), 4.55 (t, J=6.4 Hz, 1H), 4.39-4.29 (m, 2H), 4.25-4.13 (m, 2H), 3.93-3.83 (m, 1H), 3.81-3.74 (m, 1H), 3.65-3.53 (m, 3H), 3.47 (t, J=5.9 Hz, 2H), 3.24 (t, J=7.6 Hz, 2H), 3.01 (s, 1H), 2.88 (s, 1H), 2.83 (d, J=6.4 Hz, 2H), 2.77-2.65 (m, 3H), 2.52 (s, 3H), 2.46 (t, J=7.1 Hz, 2H), 2.29 (s, 3H), 2.22-2.15 (m, 2H), 2.11-1.99 (m, 2H), 1.98-1.90 (m, 3H), 1.85-1.62 (m, 5H), 1.52-1.47 (m, 2H), 1.45 (d, J=7.3 Hz, 3H), 1.38 (d, J=7.2 Hz, 1H), 1.35-1.30 (m, 3H), 1.15 (m, 2H), 1.00-0.74 (m, 12H).

¹³C NMR (126 MHz, MeOD) δ 174.1, 173.9, 173.2, 172.5, 168.4, 157.0, 155.0, 153.2, 143.4, 134.3, 133.5, 128.2, 128.1, 125.0, 124.3, 121.5, 119.9, 116.4, 111.2, 61.2, 57.8, 57.6, 52.3, 50.9, 50.7, 39.7, 39.2, 39.0, 38.7, 38.6 36.5, 34.2, 29.3, 29.0, 28.9, 24.8, 24.7, 24.6, 24.1, 23.9, 22.1, 20.1, 15.9, 14.5, 13.5, 9.9, 9.7.

HRMS: [M+Na⁺] calcd. for C₆₀H₈₀BF₂N₁₃NaO₁₂: 1246.5989; found: 1246.6013.

### Computational Methods

The simulation systems were built starting from the crystal structure of GzmB with PDB id 1IAU. Probes T1 and H5 were built using Maestro, and the peptide backbone of the IEPD moiety was superimposed to that of the co-crystallized inhibitor (Ac-IEPD-CHO) with the side chains and Dabcyl being manually adjusted to avoid steric clashes. Atom types for the protein and the peptide fragments of the probes were assigned using the FF14SB forcefield. The linker and the Dabcyl quenchers were parameterized using GAFF2 atom types. Three disulfide bonds were built between the Cys pairs 49-65, 142-209 and 173-208. Each system consisting of protein and probe was solvated in a truncated octahedron TIP3P water box with a buffer region of 12 Å. The necessary counterions were added to neutralize the system and a minimization stage was performed using 3,500 iterations of steepest descent with 6,500 iterations of conjugate gradient algorithms. Prior to the production runs, three independent replicates were prepared for each of the systems (i.e., Gmzb-T1 and Gzmb-H5) and each replicate was heated in three stages of 150 ps (50K to 150K, 150K to 250K and 250K to 298K) in the canonical ensemble using a timestep of 1 fs. Subsequently, the density was equilibrated for 500 ps in the NPT ensemble using a 2 fs timestep. A Langevin thermostat (with a collision frequency of 3 ps⁻¹) and a Montecarlo barostat were used to maintain temperature and pressure. Throughout the heating and equilibration stages, the distance between Ser 283 and the carbonyl of the Asp residue in the probes was kept under 4.0 Å using flat bottom restraints (k=5 kcal mol⁻¹ Å⁻²). The production runs for each individual replicate consisted of 200 ns long simulations in the NPT ensemble using a 2 fs timestep.

Fluorescence assays with recombinant enzymes. Fluorescence-based assays with enzymes were performed in buffer-1 (50 mM Tris, 100 mM NaCl, pH 7.4) for GzmB, pro-GzmB, GzmA and HNE, or buffer-2 (25 mM HEPES, 0.1% CHAPS, 10 mM DTT, pH 7.5) for caspases and other enzymes. Probes (25 μM) were added to enzymes (20 nM or at the indicated concentrations) in 384-well plates and their fluorescence emission was recorded at 450 nm (for AMC) or 510 nm (for BODIPY) at 37° C. using a Synergy H1 Hybrid reader (BioTek). In experiments with Ac-IEPD-CHO (50 μM), the inhibitor was pre-incubated for 1 h with GzmB before addition of any probe.

Primary cell isolation and cell culture. E0771 cancer cells expressing nuclear-localized the red fluorescent protein mKate were grown using Dulbecco's Modified Eagle Medium (DMEM) supplemented with 10% fetal bovine serum (FBS), antibiotics (100 U mL⁻¹ penicillin and 100 mg mL⁻¹ streptomycin) and 2 mM L-glutamine in a humidified atmosphere at 37° C. with 5% CO₂. Cells were regularly passaged in T-25 cell culture flasks upon reaching 90% confluency. Primary CD8+ T cells were isolated from mouse spleens by tissue homogenization, erythrocyte lysis and purification with magnetic beads (CD8 Microbeads Kit).

Computational studies and molecular dynamics simulations. Models were built with Maestro and AMBER16 using PDB id. 1IAU. The forcefields ff14SB, GAFF2 and TIP3P were used to parameterise the solvated systems. Torsional parameters for the azobenzene moiety in Dabcyl were provided by the Luque research group.⁵ For each system, 3 replicates were run at 298 K and 1 atm for 200 ns using a Langevin thermostat (collision frequency of 3 ps⁻¹), and a Monte Carlo barostat was implemented in PMEMD.⁶

Flow cytometry. E0771 (5×10⁴ cells/well) were plated on Geltrex-coated 6-well plates with IL-2 (1,000 U mL⁻¹) and co-cultured with either murine CD8+ T cells (2.5×10⁵ cells/well) which had been previously cultured for 2 days in E-DMEM with anti-mouse CD3e (2 μg mL⁻¹), anti-mouse CD28 (5 μL mL⁻¹) and IL-2 (80 U mL⁻¹). Treatments with staurosporine (1 μM) were for 1 h. Flow cytometry preparation included treatment with trypsin-EDTA (0.05%), wash, resuspension in PBS and incubation with probe H5 (5 μM) for 1 h at 37° C. Cells were then washed twice and incubated with Annexin V-AF647 (10 nM) prior to flow cytometry analysis in a 5 L LSR with data analysed with FlowJo. Excitation/emission wavelengths: H5 (488 nm, 525±50 nm), mKate (561 nm, 635±15 nm), Annexin V-AF647 (640 nm, 670±14 nm).

Live-cell fluorescence confocal microscopy. E0771 (1.5×10³ cells/well) were plated on Geltrex-coated 8-chamber glass slides with freshly isolated murine CD8+ T cells (1.2×10⁴ cells/well) in enriched E-DMEM phenol red-free media with IL-2 (80 U mL⁻¹), anti-mouse CD3e (2 μg mL⁻¹) and anti-mouse CD28 (5 μL mL⁻¹). After 48 h, T cell reinvigoration was performed by addition of IL-2 (1,000 U mL⁻¹) and incubation for 3 h. Probe H5 was used at 25 μM and Ac-IEPD-CHO at 50 μM with 1 h preincubation. After 1 h treatment at 37° C. with H5, cells were washed with media and imaged under a Leica TCS SP8 fluorescent confocal microscope under a 40× oil objective. Excitation wavelengths: 488 nm (H5), 561 nm (mKate). Images were analyzed with ImageJ.

Preclinical cancer model. SCC tumors were established and stained for flow cytometry as previously described.⁷ Briefly, 1×10⁶ SCC cancer cells were injected subcutaneously into FVB/N mice, which were sacrificed 14 days post-implantation and tumors disaggregated to generate a single cell suspension. Cells were stained with antibodies and the probe H5 for 30 min at r.t. and analysed by flow cytometry (BD Fortessa). Data analysis was performed using FlowJo software.

Drug screening. E0771 (750 cells/well) and freshly isolated murine CD8+ T cells (6,000 cells/well) were plated in 384-well plates as described above. After 2 days co-culture at 37° C., IL-2 alone (100 or 250 U mL⁻¹) or IL-2 (100 U mL⁻¹) in combination with drugs (Table 3) were further incubated for 3 h. H5 (20 μM) and Hoechst 33342 (1 μM) was added for 1 h at 37° C., washed with PBS and imaged under ImageXpress™ XLS (Molecular Devices) using KRB (10% FBS) buffer as media. Fluorescent images (4 sites/well) were acquired under a 40× objective using 405 nm (Hoechst) and 488 nm (H5) excitation wavelengths and data was analysed with MetaXpress software using the Custom Module Editor. Briefly, nuclei were used as seeds to create a pseudo-cell area and granzyme B positive puncta were detected using a top-hat filter (pixel size: 10, circle shape) followed by an average filter (3×3 pixel).

Human biopsies. Tissue samples were taken from treatment-naive patients undergoing surgical resection. Participants provided written informed consent and the study was approved by NHS Lothian REC and facilitated by NHS Lothian SAHSC Bioresource (REC No: 15/ES/0094). Fresh samples were minced, incubated with 1 mg mL⁻¹ collagenase IV, 1 mg mL⁻¹ DNase 1, 50 U mL⁻¹ hyaluronidase in DMEM for 1 h at 37° C. with agitation, passed through 100 and 70 μm filters followed by red cell lysis. For microscopy, formalin-fixed paraffin embedded slides of non-small cell lung cancer and paired non-cancerous lung were deparaffinised, rehydrated, stained with haematoxylin and eosin, followed by dehydration and imaging on a Zeiss AxioScan microscope.

### Fluorescence Assays in Urine Samples

Probe (H5 or R7) (25 μM, 50 μL) in granzyme B buffer (50 mM Tris, 100 mM NaCl, pH 7.0) was added directly to urine samples from healthy donors (50 μL) containing a range of granzyme B concentrations (20,000 pg mL⁻¹-78 pg mL⁻¹) or no granzyme B (control). The solutions were incubated at 37° C. and fluorescence response was monitored over time (t=3 h) (λ_{exc/em}: 450/510 nm or 620/660 nm). Fluorescence was read on a synergy Biotek H1 spectrophotometer.

### HPLC/MS Analysis of Probe Cleavage by Granzyme B in Urine

Probe (H5 or R7) (25 μM) was incubated with human recombinant granzyme B (15 nM) in Granzyme B buffer for 1.5 h at 37° C. Following incubation, the samples were diluted with MeOH, centrifuged and the supernatant injected into the HPLC/MS. Samples were eluted with 0.1% HCOOH in H2O (A) and 0.1% HCOOH in MBCN (B), with a gradient of 0 to 100% B over 5 minutes and an additional isocratic period of 3 minutes with a flow rate of 1.5 mL min⁻¹. Reactions were monitored from 220-700 nm and percentage cleaved was determined by comparing peak area of the cleaved probe against the intact probe on MassLynx software.

### Fluorescence Assays in Stool Samples

A high binding microplate (Merck) was coated with 50 μL (2 μg/ml) anti-GzmB antibody (human, QuickZyme Biosciences) in coating buffer (NaOAc buffer, pH 5.5) and incubated at 4° C. overnight in a humidified chamber. The plate was washed four times with wash buffer (0.01 M PBS with 0.05% (v/v) Tween 20, pH 7.5) and the antibody was blocked with addition of 100 μL of Chonblock to the 96-well plate for 1 h at room temperature. The plate was then washed with wash buffer (×4) and clinical samples diluted (1:1) in Chonblock were added to the plate (50 μL) alongside granzyme B standards which were also diluted in chonblock (1:1). Samples were incubated at room temperature for 2 h before the plates were washed with wash buffer (×4). Finally, probe H5 or R7 (25 μM) is then added to each well (50 μL total volume) and fluorescence intensity was monitored at 0 h, 2 h, 4 h, 18 and 24 h using a Synergy H1 Biotek microplate reader, where H5: λₑₓ=470 nm and λₑₘ=510 nm using monochromators at gain 60 and R7: λₑₓ=620 nm and λₑₘ=660 nm using monochromators at gain 90.

### EXAMPLES SECTION

In this work, we have rationally designed GzmB substrates and identified a new hexapeptide sequence with an optimal fit into the active site of the enzyme, suggesting an alternative binding mode for highly specific GzmB probes. The optimisation of the hexapeptide into a FRET construct resulted in the probe H5, which outperforms the k_{cat}/K_{M} ratios of IEPD tetrapeptides by some orders of magnitude. The high speed and selectivity of H5 enabled real-time measurements of the anticancer activity of T cells in a mouse model of immune-mediated tumor regression. We also demonstrated that H5 can be used in image-based screens to identify new drugs able to reinvigorate CD8+ T cells against cancer cells. Finally, we have optimised the probe H5 in biopsies from cancer patients to detect in situ T cell cytotoxic activity in human tumors.

### Design of a Highly Reactive Fluorogenic Probe for Granzyme B

The tetrapeptide IEPD has been the main scaffold reported for the preparation of covalent inhibitors as well as fluorogenic substrates targeting human GzmB (hGzmB).⁸⁻¹⁰ First, we assessed the reactivity of the commercial Ac-IEPD-AMC (i.e., a substrate that releases 7-amino-4-methylcoumaran upon reaction with hGzmB) against concentrations of enzyme that would be applicable to clinical assays. We observed slow enzymatic cleavage rates of Ac-IEPD-AMC (i.e., <1% cleavage at 20 nM enzyme for 2 h, 10% cleavage at 100 nM for 24 h) and a limit of detection (LoD) of 25 nM, which is far from the pM concentrations of GzmB found in clinical samples.¹¹ In view of these results, we examined whether IEPD-based Förster Resonance Energy Transfer (FRET) probes would show increased reactivity for hGzmB. We synthesized FRET substrates by flanking the IEPD sequence with fluorophores and quenchers as donor-acceptor pairs. Unlike in Ac-IEPD-AMC, the fluorophore was put at the N-terminal end and the quencher next to the cleavage site so that its electron-withdrawing properties might favour enzymatic cleavage. Different combinations of fluorophores, spacers and quenchers were synthesized, and the tetrapeptide T1 (with BODIPY-FL as the fluorophore and ethylenediamine-Dabcyl as the quencher) was identified as the most reactive substrate. Still, the reactivity of T1 against hGzmB was poor (i.e., <5% cleavage at 20 nM enzyme for 2 h, 15% cleavage 100 nM for 24 h) with a LoD of 17 nM.

Therefore, we decided to optimize FRET substrates by identifying sequences that could react faster and more specifically with hGzmB. Therefore, we prepared FRET hexapeptides where the IEPD sequence was extended with small amino acids at the positions P1′ and P2′ (FIG. 1a). The hexapeptides H1 (IEPDAG) and H2 (IEPDSG) showed remarkably faster conversion than the tetrapeptide T1, although incomplete conversion after 2 h with 20 nM hGzmB (e.g., 75% and 80% conversion respectively, Table 2). Since the peptide H2 showed slightly higher reactivity, we retained serine in the P1′ position and prepared the peptide H3 (IEPDSL) with a more hydrophobic leucine in the P2′ position. This change did not improve the reactivity (e.g., 80% conversion, Table 2) so we explored alternatives in the P1′ and P2′ position. The hexapeptide H4 achieved 83% conversion whereas the peptide H5 showed the highest reactivity, with fast and complete cleavage in less than 30 min (Table 2 and FIG. 5). Interestingly, this result suggested that a smaller amino acid (i.e., alanine instead of tryptophan) was better tolerated by the enzyme. To confirm this observation, we synthesized negative control peptides of the H4 and H5 sequences where we included an arginine either in P1′ or P2′ to disfavour binding. The resulting hexapeptides H6 (IEPDWR) and H7 (IEPDRL) showed markedly reduced reactivity (i.e., 40% and 60% conversion respectively, Table 2), yet still much higher reactivity than the tetrapeptide T1. These results suggest that FRET hexapeptide constructs react with hGzmB much faster than shorter tetrapeptides. We prepared all peptides using solution and solid-phase synthesis (as described below, isolated them by preparative HPLC in purities over 95% and confirmed their identity by high-resolution mass spectrometry.

All peptide conjugates were synthesized utilizing solid-phase peptide synthesis using Fmoc/Bu strategy on a 2-chlorotrityl chloride polystyrene resin. Resin loading was performed with the appropriate Fmoc protected amino acid (1.4 eq) and DIPEA (10 eq) in DCM for 1 h at r.t. Following this, the remaining 2-chlorotrityl groups were capped with MeOH (0.8 μL per mg of resin). Fmoc removal was performed with piperidine (20% in DMF) and Oxyma (1.0 M) to minimise aspartamide formation under 3×5 min cycles. Amino acids were coupled using an excess of Fmoc-protected amino acid (4 eq), COMU (4 eq), Oxyma (4 eq) and DIPEA (8 eq) in DMF for 1.5 h at r.t. Completion of the coupling was monitored by the Kaiser test (and chloranil test after coupling proline). The side chains of aspartic acid and glutamic acid were protected with OBzl groups and the arginine side chain was protected with a NO₂ group, because they can be removed under mild Pd-catalyzed hydrogenation tolerated by the acid-labile BODIPY-FL fluorophore. Fluorophore coupling was performed in solid-phase using BODIPY-FL (1.1 eq), COMU (1.15 eq), Oxyma (1.15 eq) and DIPEA (3 eq) in DMF for 1.5 h at r.t. Resin cleavage was performed by treating the resin with TFA (1% in DCM) in 5×1 min cycles. The combined solutions were then poured over DCM and evaporated under reduced pressure. Purification was performed by semi-preparative HPLC and the pure fluorescent peptides were then coupled to a Cbz-protected 1,2-diaminoethane. A catalytic hydrogenation was then performed to remove all protecting groups allowing the introduction of the quencher, Dabcyl, at the terminal amine of the linker via an NHS mediated coupling. Final peptides were isolated by semi-preparative HPLC in purities>95%.

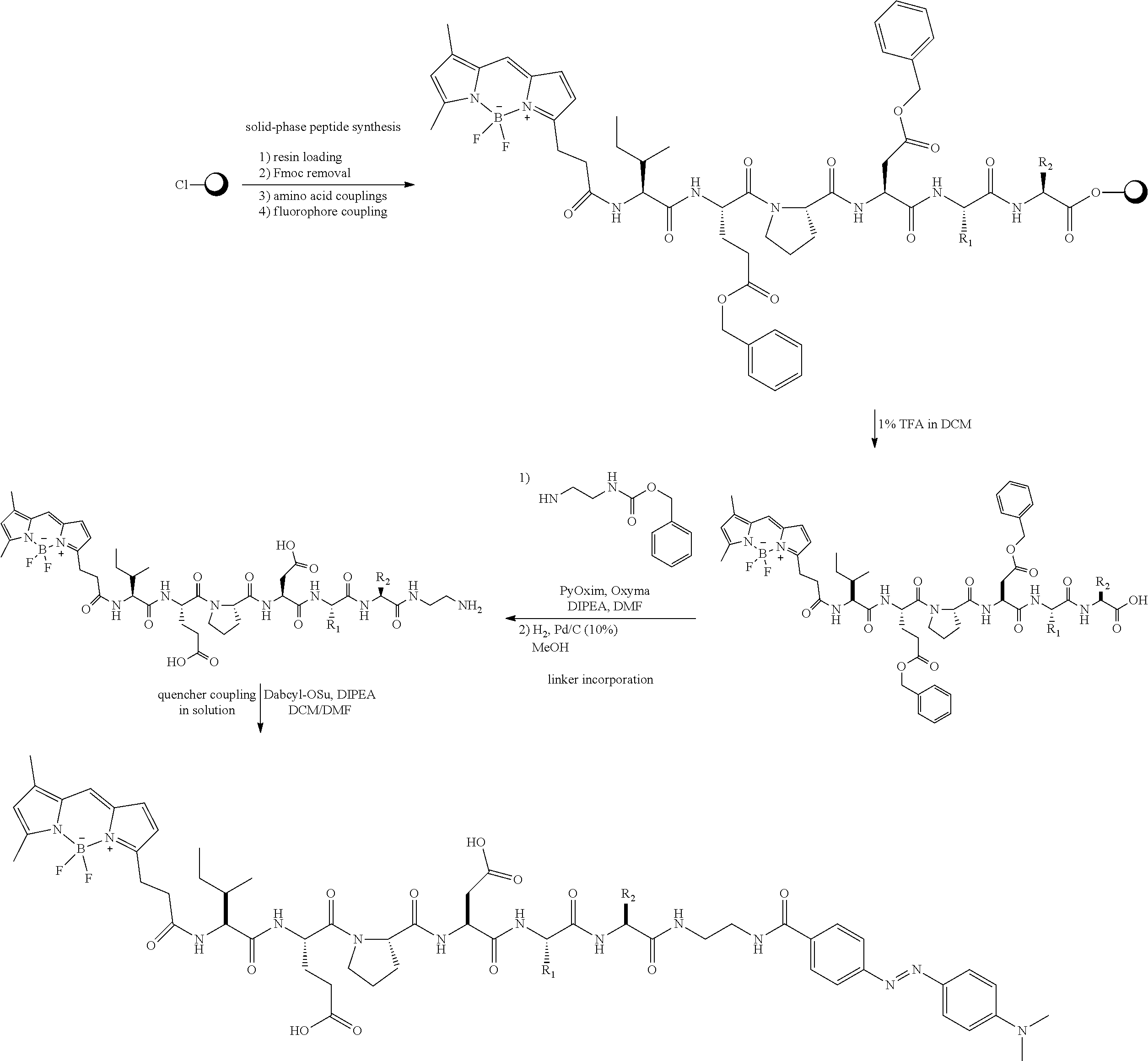

### Probe H5 is a Highly Specific Substrate of Human GzmB by Accessing a Unique Binding Pocket

We analyzed the fluorogenic response of all hexapeptides H1-H7 by measuring their fluorescence emission in the presence of recombinant hGzmB (FIG. 1a). As expected, all hexapeptides outperformed the tetrapeptide T1, with signal-to-background ratios ranging from 12-fold (for peptide H6) to 153-fold (for peptide H5) (FIG. 1b). We also measured the time taken for the most reactive compounds to reach 50-fold fluorescence increase (termed t₅₀). Probe H5 showed the fastest response among all peptides with a t₅₀ of 7 minutes (FIGS. 1b, 1c). We also compared the kinetic properties of the hexapeptide H5 to previously reported tetrapeptide constructs. The peptide H5 showed remarkably high catalytic efficiency with Vₘₐₓ values in the high μM min⁻¹ range and a k_{cat}/K_{M} ratio of 1.2×10⁷ M⁻¹s⁻¹ (FIG. 1e), which represents more than 1000-fold improvement over fluorescent tetrapeptides (Table 1). These results confirm the exceptional reactivity of probe H5, which shows an unprecedented LoD for hGzmB of 6 pM (FIG. 6b).

We also assessed the selectivity of the peptide H5 for hGzmB over other enzymes, including the inactive pro-hGzmB and other serine proteases (e.g., granzyme A, neutrophil elastase) and cysteine proteases that are active during apoptosis (e.g., caspases). The probe H5 showed minimal response to other enzymes (FIG. 1d), including caspase-3, which can cleave some IEPD-based probes (FIG. 6). This represents one important advantage when assessing response to immunotherapy, because the cross-reactivity with caspase-3 would impede distinguishing generic from T cell-mediated cancer cell death. Finally, to further confirm that the fluorescence generated from probe H5 was due to its specific reaction with hGzmB, we performed experiments in the presence of the irreversible GzmB inhibitor Ac-IEPD-CHO, which drastically reduced the fluorescence response of H5 (FIG. 1d).

In order to understand the differences in reactivity between the model tetrapeptide T1 and our new hexapeptides, we compared the preferred mode of binding of T1 and H5 in complex with a model of hGzmB using molecular dynamics (MD) simulations. Because all hexapeptides shared the BODIPY-FL and P1-P4 amino acids, we focused our analysis on the P1′-P2′ residues and the Dabcyl quencher. The MD simulations revealed that T1 and H5 accommodated the quencher in two distinct pockets. In the hGmzB-T1 simulations, the Dabcyl moiety bound preferentially to the cleft between the loop 74-84 and the loop 146-161 (FIG. 1f), whereas in the hGmzB-H5 simulations, the quencher bound preferentially to the extension of the catalytic cleft delineated by the loop 74-84 and the β-barrel 35-114 (FIG. 1f). Visual inspection of the trajectories revealed that the backbone of residues 200-201 creates a saddle point—highlighted in orange in FIG. 1f that precludes the shorter tetrapeptide T1 to accommodate the quencher along the catalytic cleft. In both cases, the Dabcyl moiety remains flexible and does not form long-live interactions with specific protein residues. The MD simulations also suggest the enhanced reactivity of H5 over the other hexapeptides is due to the combined effects of positioning an alanine residue in P1′, which fills a small hydrophobic pocket in the catalytic cleft, and a leucine residue in P2′, which straps the 200-201 saddle point (FIG. 1g). This binding mode explains the enhanced reactivity of peptide H5 over 1) peptides H4 and H6, because tryptophan in P1′ is too large to fit in the pocket, 2) peptide H7, whose arginine in P1′ is electrostatically disfavoured by the basic residues that delineate this sub-pocket, 3) peptide H3, whose serine in P1′ lacks neighbouring effective hydrogen-bond donors, and 4) peptides H1 and H2 because the glycine in P2′ is too small to pack effectively against the 200-201 saddle point.

Probe H5 Detects Real-Time Reinvigoration of T Cells in Co-Cultures with Cancer Cells

Given the reactivity and selectivity of probe H5 for GzmB, we studied its utility to measure the cytotoxic activity of T cells during the attack to cancer cells. To investigate this, we co-cultured mouse CD8+ T cells and E0771 breast cancer cells (FIG. 2a). Substrates for mouse GzmB feature a phenylalanine in P2—instead of proline in human substrates —, yet we observed good response of the probe H5 to mouse GzmB, with slightly better reactivity than the hexapeptide analogue containing a phenylalanine (probe H5ₘ: IEFDAL, FIG. 7). Therefore, we decided to use the probe H5 for both mouse and human assays. First, we optimized the co-cultures to effectively increase the levels of GzmB in CD8+ T cells. Incubation with interleukin IL-2 produced the largest reinvigoration, with over 80% CD8+ T cells expressing inactive GzmB after IL-2 treatment=and confirmation by fluorescence microscopy using anti-GzmB (FIG. 2b). Next, we applied these conditions to co-cultures of CD8+ T cells and genetically-modified E0771-La2-NLR cells, which express the red fluorescent protein mKate to facilitate their detection, and incubated them with probe H5 and Annexin V-AF647, a marker of apoptosis. Flow cytometric analysis revealed that >80% of cancer cells that had been cultured with activated T cells were double-stained with H5 and Annexin V-AF647, confirming that the emission of probe H5 indicates immune-mediated cancer cell death (FIG. 2c). Co-cultures of cancer cells with non-reinvigorated T cells or cancer cells treated with staurosporine—a kinase inhibitor that induces apoptosis—showed weak fluorescence, confirming that probe H5 detects dead cancer cells that have been killed by active CD8+ T cells (FIG. 2c). Fluorescence microscopy experiments also corroborated that probe H5 only stained the cytoplasm of mKate+ cancer cells, but not T cells (FIG. 2d). As additional controls, we confirmed that probe H5 was not cytotoxic (FIG. 8) and did not stain cancer cells in co-cultures where T cells were inactive or in co-cultures that had been pre-treated with the irreversible GzmB inhibitor Ac-IEPD-CHO (FIG. 2d). Altogether, these results confirm that probe H5 can detect cancer cell death resulting from the attack of invigorated CD8+ T cells and suggests that the fluorescence emission of probe H5 can be used as a biomarker of immunomodulatory efficacy in live cultures.

Next, we examined whether H5 could be used to image how CD8+ T cells attack cancer cells in real time. We utilized CD8+ T cells expressing the OT-I transgenic receptor, which specifically targets cells presenting the OVA-derived SIINFEKL antigen in the context of H-2 kb and co-cultured them with SIINFEKL-pulsed EL4 cancer cells (FIG. 9). Cells were counterstained with Cell Tracker Orange for ease of identification and incubated with probe H5 and the cell death marker Sytox Blue before time-lapse microscopy. Initially, the probe H5 was silent but its emission inside target cells gradually increased as CD8+ T cells started to form immune synapses with cancer cells (FIG. 2e and). After several contacts between CD8+ T cells and cancer cells, the green fluorescence signal of H5 co-localized with the blue signal of Sytox Blue, indicating that targeted cancer cells had undergone GzmB-mediated apoptosis. Quantitative flow cytometric analysis also confirmed that the fluorescence signal of the peptide H5 in cancer cells preceded that of Sytox Blue, which corroborates active GzmB as an early biomarker of immune-mediated cancer cell death (FIG. 2f). In parallel, we performed in vitro experiments mimicking the intracellular environments found in cancer cells that receive multiple contacts from CD8+ T cells and observed an accumulative fluorogenic signal for probe H5 to spiked 1 nM increments of GzmB (FIG. 9).

### Probe H5 Detects T Cell-Mediated Tumor Regression in a Mouse Model of Cancer

We further investigated the capability of the peptide H5 to detect GzmB activity in a mouse model of tumor regression. Serrels et al. reported that inhibition of Focal Adhesion Kinase (FAK) can drive T-cell mediated regression of squamous cell carcinoma (SCC) tumors via modulation of the immuno-suppressive microenvironment (FIGS. 3a, 3b), with FAK inhibitors currently being in clinical trials in combination with immune checkpoint inhibitors.¹² Because the tumour regression in SCC FAK (−/−) mice is dependent on CD8 T+ cells, this preclinical model represented an excellent platform to examine whether the probe H5 could detect T cell-mediated cancer cell death in tumors.

First, FVB immune-competent mice were challenged with SCC FAK (−/−) cancer cells or wild-type SCC cancer cells (as a negative control), and tumors were grown for 2 weeks. As expected, SCC FAK (−/−) tumors were significantly smaller and contained less live cells (FIG. 3c) than wild-type SCC tumors. The number of CD8+ T cells was around 10-fold higher in SCC FAK (−/−) tumors, implying an invigorated T cell response (FIG. 3d), which also showed higher expression levels if GzmB as determined by ELISA (FIG. 3j). In order to evaluate whether the cancer cells in SCC FAK (−/−) tumors contained intracellular active GzmB, tumors were harvested and treated with the probe H5 for 30 min before being analyzed by flow cytometry. Notably, over 40% of cancer cells in the SCC FAK (−/−) tumors were stained with probe H5, whereas wild-type SCC tumors were almost not stained (FIGS. 3e, 3f). Furthermore, we observed that the fluorescence signal of the probe H5 was exclusively found in a subset of cancer cells but not in other cells (e.g., monocytes, fibroblasts, CD4+ T cells) found in tumors (FIGS. 3g, 3h and 3k). Altogether, these results corroborate the utility of probe H5 to rapidly detect T cell-mediated cell death in mouse tumors.

### Probe H5 Identifies Immunomodulatory Activity in Drug Screens and Human Tumor Biopsies

We next assessed the utility of the probe H5 in screens of immunomodulatory drugs and clinical assays in human tumor biopsies (FIG. 4a). First, we adapted our co-cultures of mouse CD8+ T cells and E0771 cancer cells to a 384 well-plate format for image-based phenotypic assays that could screen small molecules for invigorating the killing capacity of CD8+ T cells. We used the ImageXpress high-content analysis system to test a collection of anticancer drugs with varied pharmacological targets (Table 3). E0771 and CD8+ T cells were co-cultured for 2 days prior to incubation with IL-2 (100 U mL⁻¹) and each individual drug at their respective working concentrations (Table 3). One hour before imaging, the probe H5 was added to the wells and we acquired fluorescence microscopy images, including Hoechst 33342 as a nuclear counterstain. The fluorescence intensity of probe H5 inside cancer cells was used to compare the immunomodulatory capacity of all 44 drugs. We included wells with a high concentration of IL-2 (250 U mL⁻¹) as a positive control for high GzmB and wells with 100 U mL⁻¹ IL-2 plus rapamycin, a known mTOR inhibitor that blocks IL-2-induced activation of T cells, as a negative control for low GzmB activity. Of note, a small set of compounds with different pharmacological function (e.g., protein kinase inhibitors, inhibitors of microtubule function, DNA alkylating agents, proteasome inhibitors) exhibited superior staining than the single treatment with IL-2, indicating ability to invigorate anticancer T cell activity.

Increased fluorescence signals (compared to IL-2 only controls) were observed for different combinations of IL-2 with small molecules. Among these, we identified compounds with different mechanisms of action. AZD5363 (C5) is a protein kinase AKT inhibitor; docetaxel (C11), ARQ-621 (C12) and epothilone B (C13) are direct inhibitors of microtubule function; mitomycin C (C18) and temozolomide (C20) are DNA alkylating agents and lactacystin (C30) is an irreversible proteasome inhibitor. Some of these compounds are already approved for medical use as anti-cancer drugs. Docetaxel (Taxotere®), mitomycin C (Mutamycin®) and temozolomide (Temodar®) are well-established chemotherapy drugs for the treatment of several types of cancer, including, breast (docetaxel), lung (mitomycin) and brain tumors (temozolomide). Other compounds are currently being tested in clinical trials. For example, AZD5363, alone or in combination with other drugs (e.g. paclitaxel), is currently in phase II studies for patients with metastatic breast or gynecological cancer ARQ 621 has been evaluated in phase I trials for patients with late-stage solid tumors or hematologic malignancies. Similarly, epothilone B is in phase II studies for patients with advanced colorectal, kidney and prostate cancers, among others.

Among these, the AKT kinase inhibitor AZD5363 exhibited the brightest H5 fluorescence staining and was selected for further studies. We analyzed the cell viability and extent of H5 fluorescence staining in E0771 cancer cells that had been incubated with AZD5363 as well as in CD8+ T cell plus E0771 cell co-cultures that were incubated with IL-2 only or IL-2 plus AZD5363. The treatment of AZD5363 on its own did not induce significant cancer cell death nor cause H5 staining whereas the same concentration of AZD5363 in combination with 100 U mL⁻¹ IL-2 caused significant cancer cell death and H5 fluorescence emission in CD8+ T cell-E0771 cell co-cultures (FIG. 10). These observations are in agreement with recent reports, which suggest that AKT inhibition may prevent CD8+ T cell exhaustion, resulting in enhanced cytolytic activity against target cells. These results highlighted the potential of AZD5363 to invigorate the anticancer activity of CD8+ T cells when used in combination with IL-2 and demonstrate the validity of our H5-based imaging screen platform for the identification of new immunomodulatory drugs.

Finally, we examined whether probe H5 could also monitor cytotoxic T cell function in biopsies from lung cancer patients as a method to screen their potential predisposition to anticancer treatments. For these experiments, we obtained paired (i.e., healthy vs cancerous, FIG. 4c) tissue resections from treatment-naïve lung cancer patients undergoing surgical resection. In order to analyze whether probe H5 could detect differences in CD8+ T cell reinvigoration between healthy and cancerous regions, we treated all samples with probe H5 and promptly analyzed them by flow cytometry together with an antibody-based panel of epithelial and immune cell markers. As shown in FIG. 4d, we observed significant differences in the percentages of EpCAM+H5+ cells between healthy and cancerous tissue across of all patients, indicative of the activated state and cytotoxic activity of CD8+ T cells against lung epithelial cells in the tumor microenvironment. Further optimization studies will be needed to adapt probe H5 for the evaluation of immunotherapy regimes in cancer patients. Altogether, our results indicate that probe H5 can be used in vitro in high-throughput screening assays to facilitate the discovery of new immunotherapy combinations as well as in the clinical characterization of human tumor biopsies, opening new avenues to accelerate the development of personalized anticancer immunotherapies.

### CONCLUSIONS

We described the rational design of a fluorogenic probe for rapid detection and imaging of active mouse and human GzmB. Starting from the generic IEPD sequence, we have built a collection of FRET probes to optimise the hexapeptide H5 with an unprecedented k_{cat}/K_{M} ratio of 1.2×10⁷ M⁻¹s⁻¹ and a limit of detection of 6 pM. We have used molecular dynamic simulations to investigate the binding modes of the peptides and observe that H5 binds to an extension of the catalytic cleft delineated by the loop 74-84 and the β-barrel 35-114 of hGzmB, which is inaccessible by tetrapeptide sequences. The discovery of this alternative binding mode to active hGzmB might facilitate the design and optimisation of imaging probes and enzyme inhibitors. We have demonstrated that the fluorescence emission of H5 can be used as a direct reporter of immune-mediated tumor killing ability in live cultures of CD8+ T cells and cancer cells, both qualitatively by fluorescence microscopy and quantitatively by flow cytometry. Importantly, probe H5 does not fluoresce inside T cells—where GzmB is inactive- or in cancer cells when killed by other agents (i.e., staurosporine) that are not related to immune-mediated cancer cell death. We have also shown that the probe H5 can distinguish between mouse tumors undergoing immune-mediated regression in a model of squamous cell carcinoma and identify small molecule drugs invigorating immunomodulatory responses in image-based phenotypic screens. Finally, we have used H5 for the clinical characterization of human tumour biopsies, highlighting a potential application for the personalised detection of early responses to anticancer immunotherapies.

### Near Infrared Granzyme B Probe Synthesis

In order to further develop the granzyme B technology we sought to develop Near-infrared (NIR) emitting smart probes for the detection of active granzyme B. The use of NIR light presents several advantages over shorter wavelength emission—such as a greater depth penetration through skin, decreased autofluorescence and minimal light scattering—thus enhancing signal to noise in biological samples.

Based on the work described above in developing GzmB specific sequences we intended to retain a particularly efficacious hexapeptide sequence that confers fast reactivity and selectivity with GzmB (Ile-Glu-Pro-Asp-Ala-Leu) as seen with probe H5. To produce the NIR GzmB smart probes it is necessary to swap not only the fluorophore but also the quenching group in order to retain the FRET process which renders the probe non-fluorescent in the absence of GzmB. Changes in smart probe constituents can reduce reactivity with the desired target therefore we sought to minimize this risk by developing 12 NIR emitting smart probes utilising combinations of 2 different NIR fluorophores (Silicon Rhodamine and Sulfo-Cy5), 2 linker types (hydrophobic and hydrophilic) and 3 different NIR quencher groups (QSY21, BXL-670 and BHQ-3). Chemical structures are shown below (note the chemical structure of QXL-670 has not been disclosed and is not available).

Fluorophores

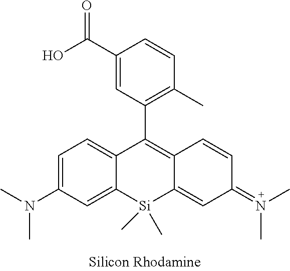

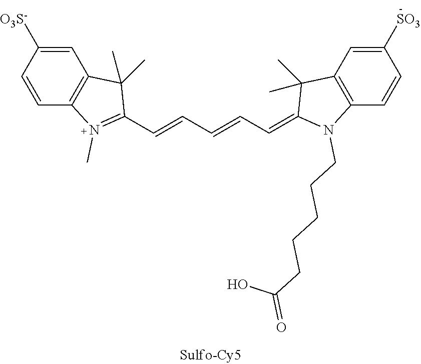

Linkers

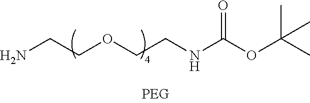

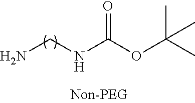

Quenchers

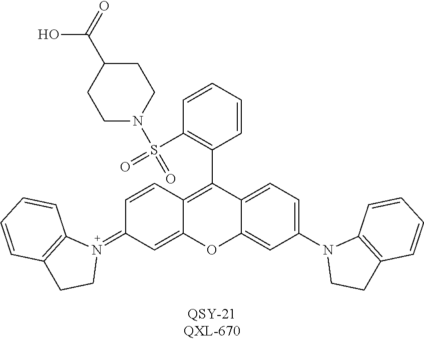

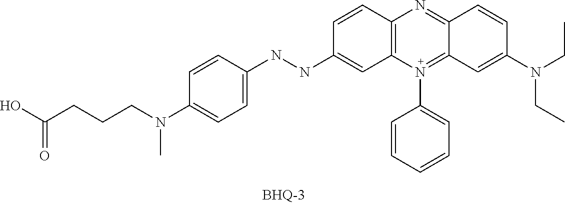

Chemical structures of the fluorophores, linkers and quenchers (except for QXL-670) used in the development of the NIR probes for detecting Granzyme B activity.

The synthetic strategy toward the NIR smart probes begins with solid phase peptide synthesis on a 2-chlorotrityl linked polystyrene resin, here we employed an Fmoc/^{t}Butyl strategy with Asp(O^{t}Bu) and Glu (O^{t}Bu) as the only residues requiring side-chain protection. The O^{t}Bu side chain protecting group was utilised owing to its orthogonality with Fmoc deprotection and peptide cleavage from the resin. Each coupling utilised COMU and Oxyma as coupling reagents and a 20% piperidine, 1M Oxyma in DMF solution for Fmoc removal (Oxyma was added to minimize aspartamide side product formation during Fmoc removal). Each of the two fluorophores (see above) were coupled last prior to removing the peptide from the resin with TFA (1% in DCM), thus rendering SiRho and SulfoCy5.

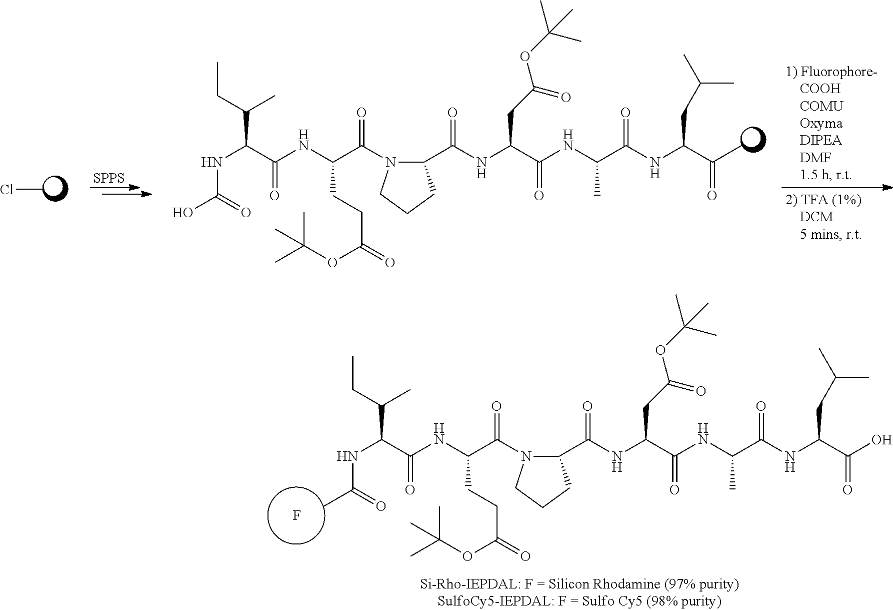

Solid-phase peptide synthesis of Silicon Rhodamine and SulfoCy5 containing Granzyme B hexapeptides.

Next, the linkers were incorporated at the free C-terminus of the peptide. We chose two linkers to utilise, namely, N-Boc-1,6-hexanediamine and tert-Butyl (14-amino-3,6,9,12-tetraoxatetradecyl)carbamate. Each of these linkers display contrasting polarity and were chosen to monitor their effect on substrate cleavage by GzmB. They were both mono-boc protected in order to free the terminal amine after side chain butyl removal as boc removal also requires a high percentage of TFA. Linker conjugation was achieved by reacting the free C-terminus of the peptides with the respective linker in the presence of PyOxim, Oxyma and DIPEA in a mixture of DCM/DMF (1:1) for 1.5 h at −20° C. The crude mixtures were purified by reverse phase semi-preparative HPLC.

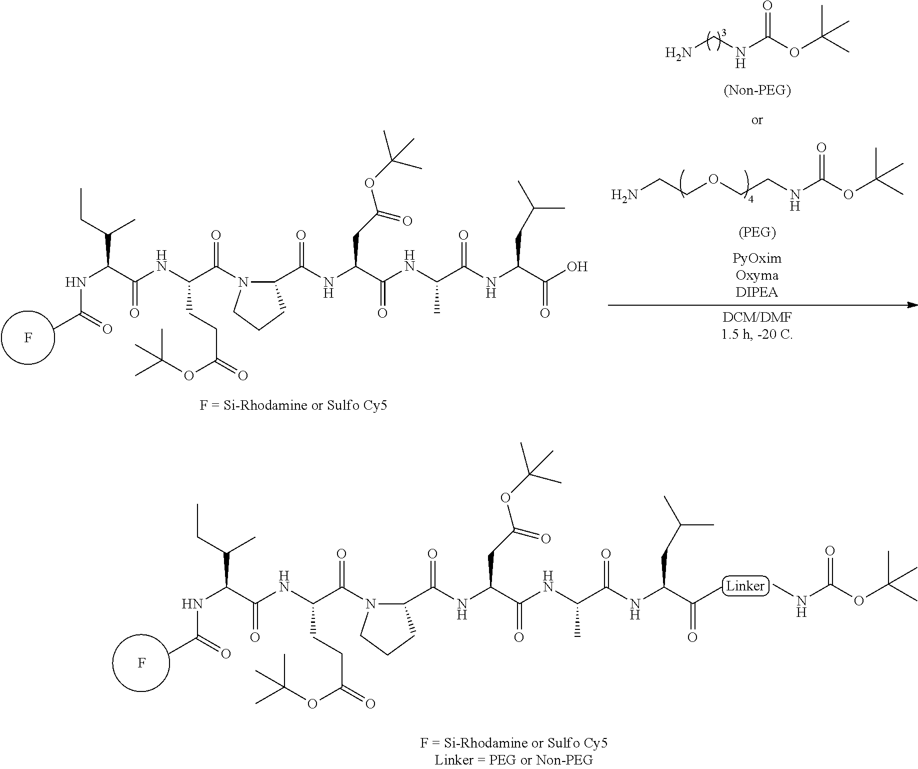

Linker conjugation to the fluorophore-peptide reagents using PyOxim and Oxyma as coupling reagents.

The next step in the sequence involved removing the t-butyl protecting groups from the side chains of the glutamic and aspartic acid alongside the boc protected amine for all of the above probes. This was done by dissolving the probes in DCM and treating with TFA (50%) for 1 h at room temperature. The solvents were then removed under reduced pressure and the amorphous solid remaining was purified by washing with diethyl ether.

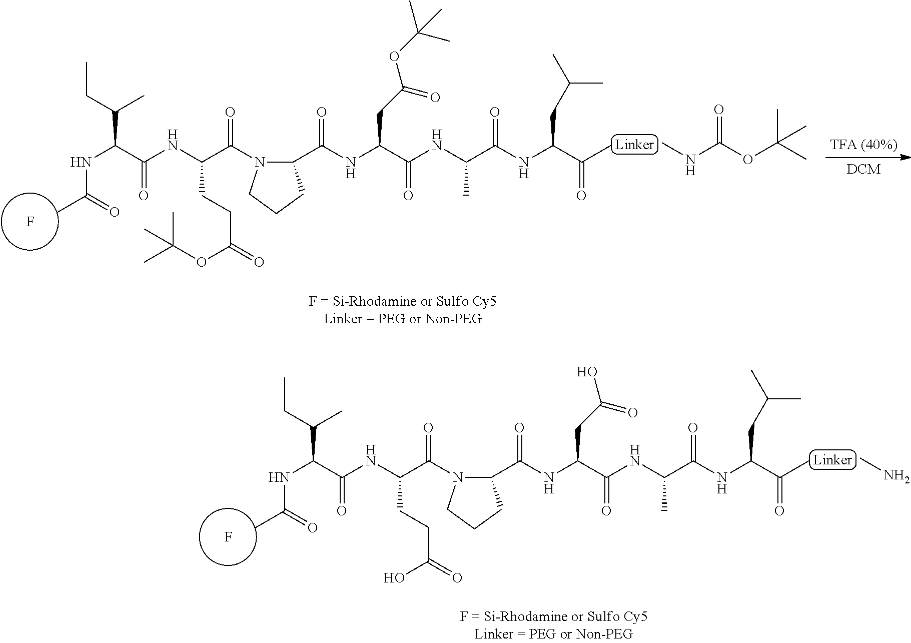

Protecting Group Removal with TFA (40%) in DCM for all Fluorophore-Peptide-Linker Conjugates

The final step in the synthesis of the full NIR probes was the addition of the quencher moiety. Each quencher was purchased as a succinimide ester allowing for conjugation to the terminal amine of the linker after deprotection. The succinimide ester is sufficiently activated to not require coupling reagents to form the amide bond with the terminal amine, thus avoiding potential side reactions with the side chain carboxylic acid groups of the peptide sequence. To each of the fluorophore-peptide-linker conjugates was added each of the 3 quenchers (QXL-670, QSY21 or BHQ-3) in separate reactions. DIPEA was then added to facilitate the amide bond formation and the reactions were stirred at room temperature for 2 days. Upon reaction completion (as monitored by HPLC) the crudes were purified by reverse-phase semi preparative HPLC to yield the final probes as blue amorphous solids (Table 4).

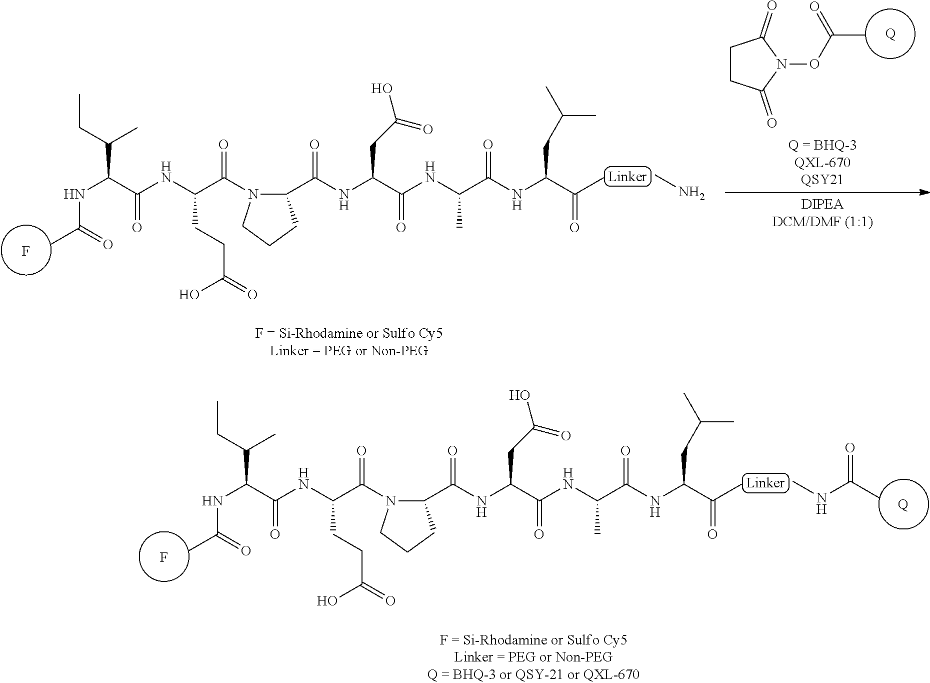

Quencher addition to the Fluorophore-peptide-linker conjugates.

Reactivity of NIR Probes with Granzyme B

With the probes in hand (Table 4) they were tested for their reactivity with human recombinant granzyme B to identify a lead candidate. Each probe was tested at a concentration of 25 μM against 20 nM granzyme B and fluorescence was recorded over 2 hours at 37° C. (λₑₓ: 620 nm, λₑₘ: 660 nm). Results demonstrated that probe R7 performed best with a fluorescence fold change of ˜80 at 660 nm following addition of granzyme B (FIG. 14 A). HPLC-MS was used to confirm the reactivity of R7 with granzyme B and it showed that conversion was 100% complete from intact to cleaved within 2 hours and that the probe was cleaved after the aspartic acid residue (FIG. 14 B). Next we investigated the selectivity of R7 for granzyme B by incubating it with closely related proteases, namely—Caspase-3 and Granzyme A. We observed no increase in fluorescence at 660 nm whilst we further confirmed the specificity of R7 for granzyme B as the fold change decreased upon pre-incubation of granzyme B with a known commercial inhibitor (Ac-IEPD-CHO) (FIG. 14 C). Finally, we investigated the kinetics of R7 consumption by human recombinant granzyme B by varying substrate concentration and calculating initial rates with a fixed concentration of enzyme (20 nM) (Kinetic at 25 μM substrate displayed in FIG. 14 D). From these results we determined that R7 has a K_{cat}/K_{M} of approx. 1.98×10⁶ M⁻¹ s⁻¹.

Optimisation of Assay Conditions (with Probe H5)

We compared the performance of probe H5 in different buffer compositions and optical settings using various concentrations (0, 8.4, 28, 84, 280, 840, 2800, 8400, 28000, and 84000 pg/ml) of hGzmB (as stated in Table 5) to establish the optimal settings for the probe. Unless otherwise stated in Table 5, 25 uM of H5 was mixed with hGzmB in a 384-well black microplate and incubated at 37° C. before quantifying fluorescence intensity. The probe concentration, ionic strength and pH of the buffer, and the optical settings on the Synergy H1 Biotek microplate reader, were modified to obtain the lowest LoD possible (Table 5).

Antibody Capture Activity Assay (with Probe H5)

As antibodies are highly specific to their substrate and are commonly used to capture material from samples (particularly those with complex matrices) in immunoassay techniques, an additional antibody capture step was added to detect GzmB in buffer and biological samples (e.g. serum). A high-binding microplate (Merck) was coated with an anti-GzmB antibody in coating buffer and incubated at 4 C overnight in a humidified chamber. The plate was washed four times with wash buffer (0.01 M PBS with 0.05% (v/v) Tween 20, pH 7.5), and 100 ul active GzmB standards, diluted from 1 mg/ml stock to various concentrations in GzmB buffer (NaCl 100 mM, Tris 50 Mm, pH 7), were added and incubated for 1 h with shaking at 50 rpm. The samples were again washed four times with wash buffer and 100 ul 25 uM probe H5, diluted from 10 mM stock in optimised GzmB buffer, was added before reading fluorescence intensity at 0 h, 2 h, 4 h, and 24 h using the Synergy H1 Biotek microplate reader, where λₑₓ=470 nm and λₑₘ=510 nm using monochromators. Fluorescence intensity was plotted against GzmB concentration, and the limit of detection (LoD) was calculated based upon the mean plus 3 standard deviations of the zero concentration sample. Other ligands (e.g. proteins, nanobodies) could be used to capture the enzyme to the surface of the microwell plate, such as described above.

The LoD obtained in serum was 40.9 pg/ml, which is comparable to pathological levels of GzmB in blood, thus could have excellent potential to be used with clinical samples.

### Testing Urine Samples for Acute Transplant Rejection

### Background

The gold standard investigation for detecting acute transplant rejection involves taking a sample of material from the transplanted organ, called a core biopsy. However, this is an invasive test which can cause harm to patients and for one in ten cases does not provide adequate tissue for diagnosis. Granzyme B (GzmB) is a protein associated with acute transplant rejection and the measurement of its activity can identify the early stages of rejection with high accuracy.

Kidney transplantation remains the single most effective treatment for end-stage kidney failure. Around one in five patients will experience acute rejection within the first year of receiving a renal transplant and is associated with an increased risk of graft loss and death. Therefore, the early detection and effective treatment of acute transplant rejection is critical to maximise graft function and quality of life for patients.

GzmB can be present in urine, but current methods to determine this require expensive equipment and sample processing. Herein we address this issue by applying probe H5 and R7 to urine samples spiked with recombinant granzyme B to detect enzyme activity.

### Results

We first sought to address the stability of probe H5 and R7 and in urine from 3 healthy donors to ensure no cross-reactivity occurred with any other biomolecules that may be present in all urine samples. To do this we monitored the fluorescence response of probe H5 and R7 in urine with and without spiked granzyme B (15 nM) and also assessed stability via HPLC analysis. Results indicated a 7-fold increase fluorescence increase with probe H5 at 510 nm and a 90-fold increase with probe R7 at 660 nm, whilst HPLC analysis reveals that less than 1% of both probes had been cleaved whilst in the urine sample alone and up to 84% and 99% conversion to the cleaved probe occurred for H5 and R7 (respectively) after 1 h with granzyme B present. (FIG. 11). It should also be noted that HPLC-MS confirmed the cleavage of probe H5 and R7 after the aspartic acid residue (the natural granzyme B cleavage site).

Next, we looked at detecting spiked recombinant granzyme B within 3 healthy urine samples to determine a limit of detection. We added a dynamic range of concentrations (20,000-78 pg/mL) of active recombinant granzyme B to the healthy urine samples and then incubated the samples with probe H5 and R7 (25 μM) for 3 hours at 37 C. We obtained an LoD of 6.18 pM (198 pg/mL) for probe H5 and 3.1 pM (99 pg/mL) for probe R7.

### Testing IBD Patient Stool Samples for Granzyme B Activity

### Background

Inflammatory Bowel Diseases (IBD) are chronic immune-mediated disorders causing inflammation of the gastrointestinal tract. In the past decade, IBD has emerged as a public health challenge, with 3 million people suffering from the disease in the EU, and a global prevalence exceeding 0.3%. The most common cases are Crohn's disease and ulcerative colitis (UC).

Despite progress in the diagnosis and treatment of IBD, new biomarkers of IBD are necessary to refine how we monitor gut inflammation, the response to treatment and choice of therapy.

Current monitoring of IBD is performed in the form of colonoscopies (an invasive procedure with waiting times of several months), histological analysis of biopsies or by measuring the abundance of limited proteins in blood or stool samples deposited at a clinic with results being ready in 2 or 3 weeks. Colonoscopies are invasive and expensive; whilst imaging scans (e.g., Magnetic Resonance Imaging (MRI), Computed Tomography (CT)) do not provide direct assessment of the gut mucosa. Mucosal healing has been used to assess the efficacy of treatments in IBD patients, but it relies heavily on histology of invasive biopsies over long periods of time.

Some biomarkers of IBD already exist, such as C-reactive protein and calprotectin and these can be detected in blood/stool samples. However, these biomarkers are generic indicators of inflammation and cannot specifically report on IBD activity. Thus, it would be good to have additional or more specific markers at the clinician's disposal.

Granzyme B is directly implicated in inflammatory diseases and is highly active during chronic gut inflammation, as shown in studies with serum of IBD patients (R. Kalla et al., J. Crohn's. Colitis. 2020, 15, 699-708) and as seen in gut tissue biopsies. Very high levels of active Granzyme B can damage the gut and become one of the reasons why inflammation does not resolve. The guts of IBD patients have high infiltration of immune cells (T Cells) capable of inducing intestinal damage in a Granzyme B-dependent manner. Development of the first IVD for Granzyme B using our fluorescent chemical probes may disrupt the current process of IBD diagnosis and monitoring via colonoscopy. Herein we utilise probe H5 and R7 in clinical stool samples based on calprotectin levels to establish if granzyme B activity can also differentiate between healthy and disease.

### Results

We used our in-house developed antibody capture assay to assess the activity of granzyme B within each stool sample (FIG. 12). Firstly, a high binding 96-well plate was coated with granzyme B antibody for 2 h at 37° C. The bound antibody was then blocked using Chonblock (2 h at r.t.) and washed prior to addition of the clinical stool samples, which were incubated at room temperature for 2 h. The plate was then washed and probes H5 and R7 were added with fluorescence being monitored over 18 h at 37° C. In order to quantify the granzyme B signal detected we included a calibration curve on each 96-well plate where a known concentration of human recombinant granzyme B was added to each well.

We analysed 48 ‘high calprotectin’ and 46 ‘low calprotectin’ stool supernatants and compared the fluorescence fold change of probe H5 and R7 in all. We quantified this fold change to give a concentration value aligning with the calibration curve (FIGS. 13 A and B). Quantifying this gave an average concentration of 4802 pg/mL and 11636 pg/mL for low calprotectin and high calprotectin samples with probe H5, respectively (FIG. 13 A). Meanwhile, we noted an average concentration of 3318 pg/mL and 5687 pg n mL for low calprotectin and high calprotectin samples with probe R7, respectively (FIG. 13 B).

TABLE 1
Summary of fluorescent tetra- and hexapeptide probes for GzmB.

| | | Amplification | k_{cat} | K_{M} | k_{cat}/K_{M} | | | |
|---|---|---|---|---|---|---|---|---|
| | Compound | mechanism | (s⁻¹) | (μM) | (M⁻¹ s⁻¹) | LoD | | Ref. |
| 4-mer | Ac-IEPD-AMC | Fluorogenic dye | 0.5 | 160 | 3.3 × 10³ | 25 | nM | Ref. 10 |
| | PEGylated IONPs | Nanoconstruct | ~0.002 | ~0.2 | 1.1 × 10⁴ | n.d. | | Ref. 3 |
| | CyGbP_{F} | Fluorogenic dye | 0.07 | 22.7 | 3.1 × 10³ | n.d. | | Ref. 4 |
| | T1 | FRET | 2.3 | 30.8 | 5.9 × 10⁴ | 17 | nM | this work |
| 6-mer | H5 | FRET | 117 | 9.6 | 1.2 × 10⁷ | 6 | pM | this work |

TABLE 2
Sequences and reactivity of FRET-based hexapeptides against hGzmB.

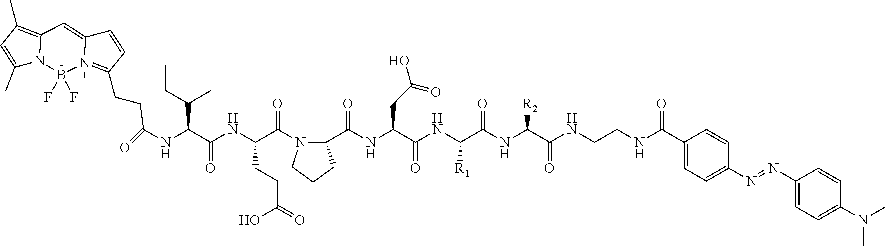

| | | HPLC conversion |
|---|---|---|
| Code | Peptide sequence | (20 nM hGzmB, 2 h) |
| H1 | Ile-Glu-Pro-Asp-Ala-Gly | 75% |
| H2 | Ile-Glu-Pro-Asp-Ser-Gly | 80% |
| H3 | Ile-Glu-Pro-Asp-Ser-Leu | 80% |
| H4 | Ile-Glu-Pro-Asp-Trp-Leu | 82% |
| H5 | Ile-Glu-Pro-Asp-Ala-Leu | 100% |
| H6 | Ile-Glu-Pro-Asp-Trp-Arg | 40% |
| H7 | Ile-Glu-Pro-Asp-Arg-Leu | 60% |
| H5m | Ile-Glu-Phe-Asp-Ala-Leu | 70% |

TABLE 3
Screened small molecule drugs and working concentrations.

| | | Conc. |
|---|---|---|
| Code | Compound | (μM) |
| C1 | Dinaciclib | 1 |
| | (CDK inhibitor) | |
| C2 | Seliciclib | 1 |
| | (CDK inhibitor) | |
| C3 | Genistein | 10 |
| | (tyrosine kinase inhibitor) | |
| C4 | Trametinib | 0.1 |
| | (MEK inhibitor) | |
| C5 | AZD5363 | 1 |
| | (AKT inhibitor) | |
| C6 | Saracatinib | 1 |
| | (Src/Abl inhibitor) | |
| C7 | Dasatinib | 0.1 |
| | (Src/Abl inhibitor) | |
| C8 | ZM447439 | 1 |
| | (Aurora inhibitor) | |
| C9 | Y27632 | 1 |
| | (ROCK inhibitor) | |
| C10 | Paclitaxel | 0.3 |
| | (microtubule inhibitor) | |
| C11 | Docetaxel | 1 |
| | (microtubule inhibitor) | |
| C12 | ARQ621 | 10 |
| | (Eg5 inhibitor) | |
| C13 | Epothilone B | 1 |
| | (microtubule inhibitor) | |
| C14 | Barasertib | 1 |
| | (Aurora kinase B inhibitor) | |
| C15 | Nocodazole | 0.3 |
| | (microtubule inhibitor) | |
| C16 | Methotrexate | 10 |
| | (DHFR inhibitor) | |
| C17 | Aphidicolin | 1 |
| | (DNA polymerase inhibitor) | |
| C18 | Mitomycin C | 1 |
| | (DNA crosslinker) | |
| C19 | Floxuridine | 10 |
| | (TS synthetase inhibitor) | |
| C20 | Temozolomide | 1 |
| | (DNA alkylating) | |
| C21 | Camptothecin | 1 |
| | (topoisomerase inhibitor) | |
| C22 | SN38 | 1 |
| | (topoisomerase inhibitor) | |
| C23 | Olaparib | 1 |
| | (PARP inhibitor) | |
| C24 | Veliparib | 1 |
| | (PARP inhibitor) | |
| C25 | Cytochalasin B | 3 |
| | (actin polymerization inhibitor) | |
| C26 | Latrunculin B | 1 |
| | (actin polymerization inhibitor) | |
| C27 | N-acetyl-leucyl-leucyl-norleucinal | 1 |
| | (calpain inhibitor) | |
| C28 | ZVAD | 1 |
| | (caspase inhibitor) | |
| C29 | N-acetyl-leucyl-leucyl-methioninal | 1 |
| | (calpain inhibitor) | |
| C30 | Lactacystin | 1 |
| | (proteasome inhibitor) | |
| C31 | Valproic Acid | 10 |
| | (histone deacetylase inhibitor) | |
| C32 | Trichostatin A | 1 |
| | (histone deacetylase inhibitor) | |
| C33 | SAHA | 10 |
| | (histone deacetylase inhibitor) | |
| C34 | Panobinostat | 0.1 |
| | (histone deacetylase inhibitor) | |
| C35 | Emetine | 1 |
| | (protein synthesis inhibitor) | |
| C36 | AZD2014 | 1 |
| | (mTOR inhibitor) | |
| C37 | Brefeldin A | 1 |
| | (Golgi apparatus breakdown) | |
| C38 | CA074Me | 3 |
| | (cathepsin B inhibitor) | |
| C39 | Simvastatin | 1 |
| | (HMG-CoA reductase inhibitor) | |
| C40 | Lovastatin | 1 |
| | (HMG-CoA reductase inhibitor) | |
| C41 | Marimastat | 3 |
| | (MMP inhibitor) | |
| C42 | Pepstatin | 10 |
| | (Aspartyl protease inhibitor) | |
| C43 | Leupeptin | 1 |
| | (Ser/Cys protease inhibitor) | |
| C44 | Aprotinin | 10 |
| | (Trypsin inhibitor) | |

TABLE 4
Characterization of Near-infrared Granzyme B probes

| | | | | HPLC t_{R} | |
|---|---|---|---|---|---|
| Code | Fluorophore | Linker | Quencher | (mins) | Purity |
| R1 | Silicon | PEG | QSY-21 | 6.67 | 96% |
| | Rhodamine | | | | |
| R2 | Silicon | PEG | QXL670 | 6.90 | 99% |
| | Rhodamine | | | | |
| R3 | Silicon | PEG | BHQ3 | 6.57 | 98% |
| | Rhodamine | | | | |
| R4 | Silicon | NonPEG | QSY-21 | 6.75 | 97% |
| | Rhodamine | | | | |
| R5 | Silicon | NonPEG | QXL670 | 7.45 | 94% |
| | Rhodamine | | | | |
| R6 | Silicon | NonPEG | BHQ3 | 6.89 | 95% |
| | Rhodamine | | | | |
| R7 | Sulfo-Cy5 | PEG | QSY-21 | 6.87 | 99% |
| R8 | Sulfo-Cy5 | PEG | BHQ3 | 6.25 | 97% |
| R9 | Sulfo-Cy5 | NonPEG | QSY-21 | 7.88 | 98% |
| R10 | Sulfo-Cy5 | NonPEG | BHQ3 | 7.38 | 99% |

TABLE 5
Various conditions that were modified to optimise the limit of detection (LoD).

| Probe concentration | | Wavelength | | NaCl | | LoD | |
|---|---|---|---|---|---|---|---|
| (μM) | Optics | (nm) | Gain | (mM) | pH | (pM) | n |
| 5 | Monochromator | 450-510 | 100 | 100 | 7.4 | 677.41 | 6 |
| 5 | Monochromator | 470-510 | 60 | 100 | 7.4 | 547.45 | 6 |
| 5 | Monochromator | 470-510 | 100 | 100 | 7.4 | 419.61 | 6 |
| 25 | Filter | 485-530 | 45 | 100 | 7.4 | 160.86 | 15 |
| 25 | Filter | 485-530 | 55 | 100 | 7.4 | 150.78 | 6 |
| 25 | Filter | 485-530 | 35 | 100 | 7.4 | 106.67 | 18 |
| 25 | Filter | 485-530 | 35 | 200 | 7.4 | 72.55 | 3 |
| 25 | Filter | 485-530 | 45 | 200 | 7.4 | 70.06 | 3 |
| 50 | Filter | 485-530 | 35 | 100 | 7.4 | 57.93 | 6 |
| 50 | Filter | 485-530 | 25 | 100 | 7.4 | 57.18 | 6 |
| 25 | Filter | 485-530 | 35 | 100 | 7.7 | 48.05 | 3 |
| 25 | Filter | 485-530 | 35 | 1 | 7.4 | 47.48 | 3 |
| 25 | Filter | 485-530 | 35 | 400 | 7.4 | 43.99 | 3 |
| 25 | Filter | 485-530 | 35 | 100 | 8 | 42.50 | 3 |
| 25 | Filter | 485-530 | 45 | 1 | 7.4 | 41.02 | 3 |
| 25 | Filter | 485-530 | 45 | 100 | 7.7 | 37.69 | 3 |
| 25 | Filter | 485-530 | 35 | 400 | 7 | 33.80 | 6 |
| 25 | Filter | 485-530 | 25 | 100 | 7.4 | 32.61 | 6 |
| 25 | Filter | 485-530 | 45 | 100 | 8 | 26.63 | 3 |
| 25 | Filter | 485-530 | 45 | 400 | 7.4 | 25.91 | 3 |
| 25 | Filter | 485-530 | 45 | 400 | 7 | 20.32 | 6 |
| 25 | Filter | 485-530 | 35 | 100 | 7 | 7.49 | 6 |
| 25 | Filter | 485-530 | 45 | 100 | 7 | 6.32 | 6 |

### REFERENCES

1. Mouchacca, P., Schmitt-Verhulst, A. M. & Boyer, C. Visualization of cytolytic T cell differentiation and granule exocytosis with t cells from mice expressing active fluorescent granzyme B. PLoS One 8, e67239 (2013).
2. Thomberry, N. A. et al. A combinatorial approach defines specificities of members of the caspase family and granzyme B: functional relationships established for key mediators of apoptosis. J. Biol. Chem. 272, 17907-17911 (1997).
3. Mac, Q. D. et al. Non-invasive early detection of acute transplant rejection via nanosensors of granzyme B activity. Nat. Biomed. Eng. 3, 281-291 (2019).
4. He, S., Li, J., Lyu, Y., Huang, J. & Pu, K. Near-infrared fluorescent macromolecular reporters for real-time imaging and urinalysis of cancer immunotherapy. J. Am. Chem. Soc. 142, 7075-7082 (2020).
5. Espargaró, A. et al. On the binding of Congo Red to amyloid fibrils. Angew. Chem. Int. Ed. 59, 8104-8107 (2020).
6. Götz, A. W. et al. Routine microsecond molecular dynamics simulations with AMBER on GPUs. 1. generalized born. J. Chem. Theory. Comput. 8, 1542-1555 (2012).
7. Serrels, A. et al. Nuclear FAK controls chemokine transcription, Tregs, and evasion of anti-tumor immunity. Cell 163, 160-173 (2015).
8. Janiszewski, T. et al. Noninvasive optical detection of granzyme B from natural killer cells with enzyme-activated fluorogenic probes. J. Biol. Chem. 295, 9567-9582 (2020).
9. Mahrus, S. & Craik, C. S. Selective chemical functional probes of granzymes A and B reveal granzyme B is a major effector of natural killer cell-mediated lysis of target cells. Chem. Biol. 12, 567-577 (2005).
10. Harris, J. L., Peterson, E. P., Hudig, D., Thomberry, N. A. & Craik, C. S. Definition and redesign of the extended substrate specificity of granzyme B. J. Biol. Chem. 273, 27364-27373 (1998).
11. Kok, H. M. et al. Systemic and local granzyme B levels are associated with disease activity, kidney damage and interferon signature in systemic lupus erythematosus. Rheumatology 56, 2129-2134 (2017).
12. Canel, M., Taggart, D., Sims, A. H., Lonergan, D. W., Waizenegger, I. C. & Serrels, A. T-cell co-stimulation in combination with targeting FAK drives enhanced anti-tumor immunity. eLife 9, e48092 (2020).

1. A probe for use in detecting granzyme B, the probe comprising a granzyme B cleavable peptide conjugated or bound to a detectable moiety, wherein the granzyme B cleavable peptide sequence consists of the sequence:
IEPDAL (SEQ ID NO:12).

2. (canceled)

3. The probe according to claim 1, wherein the detectable moiety is an isotopic- or radio-label, paramagnetic contrast agent, paramagnetic or superparamagnetic particle, or an optically detectable moiety.

4. The probe according to claim 3, wherein the optically detectable moiety is a fluorescent moiety.

5. The probe according to claim 4, further comprising a quencher moiety.

6. The probe according to claim 3 wherein the detectable moiety is conjugated to the peptide by way of a covalent bond.

7. The probe according to claim 1, wherein the detectable moiety is conjugated to the N or C terminal amino acid of the peptide and when present, a quencher moiety is conjugated to the respective C or N terminal to which a fluorescent moiety is conjugated.

8. The probe according to claim 3 wherein the detectable moiety is conjugated, bound and/or embedded within a nanoparticle, wherein the nanoparticle is conjugated to the peptide.

9. The probe according to claim 3, wherein the detectable moiety is conjugated to the peptide by way of a linker molecule.

10. The probe according to claim 9, wherein the linker molecule is an alkyl, alkenyl, or polyether chain, with optionally C2-C24 repeating units, or one or more natural or non-naturally occurring amino or imino acids.

11. The probe according to claim 1, capable of detecting granzyme B in a sample, in an amount of less than 10 nM, such as less than 1 nM, 500 pM, 250 pM, 100 pM, or less than 50 pM.

12. The probe according to claim 1, having a Km for Granzyme B of less than 30 μM, 25 μM, 20 μM, 15 μM, or 10 μM.

13. The probe according to claim 1, having a Kcat/KM value of greater than 1×10⁴, 1×10⁵, 1×10⁶, or 1×10⁷.

14. A detection device comprising the probe according to claim 1, wherein the probe is bound, adhered, or otherwise captured on a surface of the device.

15. The detection device according to claim 14, wherein the surface is a surface of a slide, microtiter plate, lateral flow device, or wall of a fluidic device.

16. The detection device according to claim 14, wherein the surface is a surface of a bead, microparticle, or nanoparticle.

17. (canceled)

18. A method of detecting Granzyme B in a cell containing sample, or bodily fluid or excretion, the method comprising contacting a probe according to claim 1, with the cell containing sample, or bodily fluid or excretion sample, or a processed portion thereof, ex vivo, in vivo, or in vitro and detecting a level of granzyme B, by cleavage of the peptide and release of a cleaved peptide comprising the detectable moiety.

19. The method according to claim 18, wherein the bodily fluid or excretion is a blood, serum, plasma, urine or lung lavage sample.

20. (canceled)

21. The method according to claim 18, wherein the bodily fluid or excretion has been processed in order to concentrate and/or isolate any Granzyme B from other components in the sample, to provide a processed sample.

22. The method according to claim 21, wherein the processed sample is contacted with a binding agent, such as an anti-Granzyme B antibody, or binding fragment thereof, which is capable of specifically binding Granzyme B, which is optionally bound, adhered or otherwise attached to a surface, prior to contacting the probe and detecting a level of Granzyme B present in the diluted stool sample.
